(19) United States
(12) Patent Application Publication
(10) Pub. No.: US 2016/0272626 A1
(43) Pub. Date: Sep. 22, 2016

(54) PYRAZINE DERIVATIVES

(71) Applicant: HOFFMANN-LA ROCHE INC., Nutley, NJ (US)

(72) Inventors: Guido GALLEY, Rheinfelden (DE); Roger NORCROSS, Olsberg (CH); Philippe PFLIEGER, Schwoben (FR)

(73) Assignee: Hoffmann-La Roche Inc., Little Falls, NJ (US)

(21) Appl. No.: 14/442,377

(22) PCT Filed: Nov. 4, 2013
(86) PCT No.: PCT/EP2013/072957

§ 371 Date: May 12, 2015

(30) Foreign Application Priority Data
Nov. 7, 2012 (EP) 12191529.2

(51) Int. Cl. C07D 413/12; C07D 413/14

(52) U.S. Cl. CPC C07D 413/12; C07D 413/14

(57) ABSTRACT

The present invention relates to compounds of formula

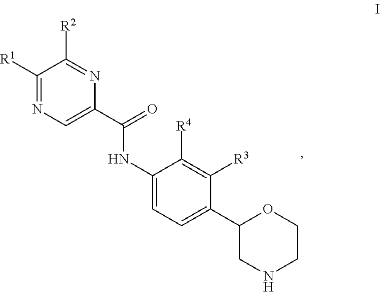

wherein
R¹/R² are hydrogen, lower alkyl, lower alkoxy, lower alkyl substituted by halogen, lower alkoxy substituted by halogen, cycloalkyl, OCH₂-cycloalkyl or heterocycloalkyl which is optionally substituted by halogen, with the proviso that one of R¹ and R² is hydrogen, or R¹ and R² form together with the carbon atom to which they are attach a phenyl ring, which may be optionally substituted by lower alkyl;
R³/R⁴ are hydrogen, halogen or cyano;
   with the proviso that one of R³ and R⁴ is hydrogen;
      or to a pharmaceutically suitable acid addition salt thereof, to all racemic mixtures, all their corresponding enantiomers and/or optical isomers, which may be used for the treatment of depression, anxiety disorders, bipolar disorder, attention deficit hyperactivity disorder (ADHD), stress-related disorders, psychotic disorders, schizophrenia, neurological diseases, Parkinson's disease, neurodegenerative disorders, Alzheimer's disease, epilepsy, migraine, hypertension, substance abuse, metabolic disorders, eating disorders, diabetes, diabetic complications, obesity, dyslipidemia, disorders of energy consumption and assimilation, disorders and malfunction of body temperature homeostasis, disorders of sleep and circadian rhythm, and cardiovascular disorders.

The present invention relates to compounds of formula

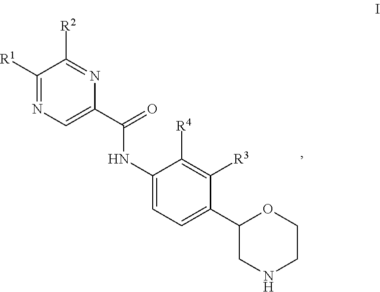

wherein
R¹/R² are hydrogen, lower alkyl, lower alkoxy, lower alkyl substituted by halogen, lower alkoxy substituted by halogen, cycloalkyl, OCH₂-cycloalkyl or heterocycloalkyl which is optionally substituted by halogen, with the proviso that one of R¹ and R² is hydrogen, or R¹ and R² form together with the carbon atom to which they are attach a phenyl ring, which may be optionally substituted by lower alkyl;
R³/R⁴ are hydrogen, halogen or cyano;

with the proviso that one of R³ and R⁴ is hydrogen;

or to a pharmaceutically suitable acid addition salt thereof, to all racemic mixtures, all their corresponding enantiomers and/or optical isomers.

It has now been found that the compounds of formulas I have a good affinity to the trace amine associated receptors (TAARs), especially for TAAR1.

The compounds may be used for the treatment of depression, anxiety disorders, bipolar disorder, attention deficit hyperactivity disorder (ADHD), stress-related disorders, psychotic disorders such as schizophrenia, neurological diseases such as Parkinson's disease, neurodegenerative disorders such as Alzheimer's disease, epilepsy, migraine, hypertension, substance abuse and metabolic disorders such as eating disorders, diabetes, diabetic complications, obesity, dyslipidemia, disorders of energy consumption and assimilation, disorders and malfunction of body temperature homeostasis, disorders of sleep and circadian rhythm, and cardiovascular disorders.

Some of the physiological effects (i.e. cardiovascular effects, hypotension, induction of sedation) which have been reported for compounds which may bind to adrenergic receptors (WO02/076950, WO97/12874 or EP 0717 037) may be considered to be undesirable side effects in the case of medicaments aimed at treating diseases of the central nervous system as described above. Therefore it is desirable to obtain medicaments having selectivity for the TAAR1 receptor vs adrenergic receptors. Objects of the present invention show selectivity for TAAR1 receptor over adrenergic receptors, in particular good selectivity vs the human and rat alpha1 and alpha2 adrenergic receptors.

The classical biogenic amines (serotonin, norepinephrine, epinephrine, dopamine, histamine) play important roles as neurotransmitters in the central and peripheral nervous system [1]. Their synthesis and storage, as well as their degradation and reuptake after release are tightly regulated. An imbalance in the levels of biogenic amines is known to be responsible for the altered brain function under many pathological conditions [2-5]. A second class of endogenous amine compounds, the so-called trace amines (TAs) significantly overlaps with the classical biogenic amines regarding structure, metabolism and subcellular localization. The TAs include p-tyramine, β-phenylethylamine, tryptamine and octopamine, and they are present in the mammalian nervous system at generally lower levels than classical biogenic amines [6].

Their dysregulation has been linked to various psychiatric diseases like schizophrenia and depression [7] and for other conditions like attention deficit hyperactivity disorder, migraine headache, Parkinson's disease, substance abuse and eating disorders [8,9].

For a long time, TA-specific receptors had only been hypothesized based on anatomically discrete high-affinity TA binding sites in the CNS of humans and other mammals [10,11]. Accordingly, the pharmacological effects of TAs were believed to be mediated through the well known machinery of classical biogenic amines, by either triggering their release, inhibiting their reuptake or by “crossreacting” with their receptor systems [9,12,13]. This view changed significantly with the recent identification of several members of a novel family of GPCRs, the trace amine associated receptors (TAARs) [7,14]. There are 9 TAAR genes in human (including 3 pseudogenes) and 16 genes in mouse (including 1 pseudogene). The TAAR genes do not contain introns (with one exception, TAAR2 contains 1 intron) and are located next to each other on the same chromosomal segment. The phylogenetic relationship of the receptor genes, in agreement with an in-depth GPCR pharmacophore similarity comparison and pharmacological data suggest that these receptors form three distinct subfamilies [7,14]. TAAR1 is in the first subclass of four genes (TAAR1-4) highly conserved between human and rodents. TAs activate TAAR1 via Gαs. Dysregulation of TAs was shown to contribute to the aetiology of various diseases like depression, psychosis, attention deficit hyperactivity disorder, substance abuse, Parkinson's disease, migraine headache, eating disorders, metabolic disorders and therefore TAAR1 ligands have a high potential for the treatment of these diseases.

Therefore, there is a broad interest to increase the knowledge about trace amine associated receptors.

### REFERENCES USED

1 Deutch, A. Y. and Roth, R. H. (1999) Neurotransmitters. In Fundamental Neuroscience (2^{nd} edn) (Zigmond, M. J., Bloom, F. E., Landis, S. C., Roberts, J. L, and Squire, L. R., eds.), pp. 193-234, Academic Press;
2 Wong, M. L. and Licinio, J. (2001) Research and treatment approaches to depression. Nat. Rev. Neurosci. 2, 343-351;
3 Carlsson, A. et al. (2001) Interactions between monoamines, glutamate, and GABA in schizophrenia: new evidence. Annu. Rev. Pharmacol. Toxicol. 41, 237-260;
4 Tuite, P. and Riss, J. (2003) Recent developments in the pharmacological treatment of Parkinson's disease. Expert Opin. Investig. Drugs 12, 1335-1352,
5 Castellanos, F. X. and Tannock, R. (2002) Neuroscience of attention-deficit/hyperactivity disorder: the search for endophenotypes. Nat. Rev. Neurosci. 3, 617-628;
6 Usdin, Earl; Sandler, Merton; Editors. Psychopharmacology Series, Vol. 1: Trace Amines and the Brain. [Proceedings of a Study Group at the 14th Annual Meeting of the American College of Neuropsychoparmacology, San Juan, Puerto Rico] (1976);
7 Lindemann, L. and Hoener, M. (2005) A renaissance in trace amines inspired by a novel GPCR family. Trends in Pharmacol. Sci. 26, 274-281;
8 Branchek, T. A. and Blackburn, T. P. (2003) Trace amine receptors as targets for novel therapeutics: legend, myth and fact. Curr. Opin. Pharmacol. 3, 90-97;
9 Premont, R. T. et al. (2001) Following the trace of elusive amines. Proc. Natl. Acad. Sci. U.S.A. 98, 9474-9475;
10 Mousseau, D. D. and Butterworth, R. F. (1995) A high-affinity [3H] tryptamine binding site in human brain. Prog. Brain Res. 106, 285-291;
11 McCormack, J. K. et al. (1986) Autoradiographic localization of tryptamine binding sites in the rat and dog central nervous system. J. Neurosci. 6, 94-101;
12 Dyck, L. E. (1989) Release of some endogenous trace amines from rat striatal slices in the presence and absence of a monoamine oxidase inhibitor. Life Sci. 44, 1149-1156;
13 Parker, E. M. and Cubeddu, L. X. (1988) Comparative effects of amphetamine, phenylethylamine and related drugs on dopamine efflux, dopamine uptake and mazindol binding. J. Pharmacol. Exp. Ther. 245, 199-210;
14 Lindemann, L. et al. (2005) Trace amine associated receptors form structurally and functionally distinct subfamilies of novel G protein-coupled receptors. Genomics 85, 372-385.

Objects of the present invention are new compounds of formula I and their pharmaceutically acceptable salts, their use for the manufacture of medicaments for the treatment of diseases related to the biological function of the trace amine associated receptors, their manufacture and medicaments based on a compound in accordance with the invention in the control or prevention of illnesses such as depression, anxiety disorders, bipolar disorder, attention deficit hyperactivity disorder, stress-related disorders, psychotic disorders such as schizophrenia, neurological diseases such as Parkinson's disease, neurodegenerative disorders such as Alzheimer's disease, epilepsy, migraine, substance abuse and metabolic disorders such as eating disorders, diabetes, diabetic complications, obesity, dyslipidemia, disorders of energy consumption and assimilation, disorders and malfunction of body temperature homeostasis, disorders of sleep and circadian rhythm, and cardiovascular disorders.

The preferred indications using the compounds of the present invention are depression, psychosis, Parkinson's disease, anxiety, attention deficit hyperactivity disorder (ADHD) and diabetes.

As used herein, the term “lower alkyl” denotes a saturated straight- or branched-chain group containing from 1 to 7 carbon atoms, for example, methyl, ethyl, propyl, isopropyl, n-butyl, i-butyl, 2-butyl, t-butyl and the like. Preferred alkyl groups are groups with 1-4 carbon atoms.

As used herein, the term “lower alkoxy” denotes a group wherein the alkyl residue is as defined above and which is attached via an oxygen atom.

The term “halogen” denotes chlorine, iodine, fluorine and bromine. The preferred halogen group is fluorine.

As used herein, the term “lower alkyl substituted by halogen” denotes a saturated straight- or branched-chain group containing from 1 to 7 carbon atoms as defined for the term “lower alkyl”, wherein at least one hydrogen atom is replaced by a halogen atom. A preferred halogen atom is fluoro. Examples of such groups are CF₃, CHF₂, CH₂F, CH₂CF₃ or CH₂CHF₂.

The term “heterocycloalkyl” denotes a non-aromatic ring with 4 to 6 ring atoms, containing at least one heteroatom, for example N, O or S. A preferred heteroatom is N. Examples of such heterocyclyl groups are azetidin-1-yl, pyrrolin-1-yl or piperidin-1-yl.

The term “R¹ and R² form together with the carbon atom to which they are attach a phenyl ring” denotes the replacement of the pyrazine group by a quinoxaline group.

The term “cycloalkyl” denotes a saturated carbon ring, containing from 3 to 6 carbon atoms, for example cyclopropyl, cyclobutyl, cyclopentyl or cyclohexyl.

The term “pharmaceutically acceptable acid addition salts” embraces salts with inorganic and organic acids, such as hydrochloric acid, nitric acid, sulfuric acid, phosphoric acid, citric acid, formic acid, fumaric acid, maleic acid, acetic acid, succinic acid, tartaric acid, methanesulfonic acid, p-toluenesulfonic acid and the like.

One embodiment of the invention are compounds of formula I, in which “halogen” is fluorine.

One embodiment of the invention are further compounds of formula I, wherein R′ is lower alkyl, lower alkoxy, lower alkyl substituted by halogen, lower alkoxy substituted by halogen, cycloalkyl, OCH₂-cycloalkyl or heterocycloalkyl, which is optionally substituted by halogen, and R² is hydrogen, for example the following compounds
(R)—N-(3-fluoro-4-(morpholin-2-yl)phenyl)-5-(trifluoromethyl)pyrazine-2-carboxamide
(R)—N-(3-cyano-4-(morpholin-2-yl)phenyl)-5-(trifluoromethyl)pyrazine-2-carboxamide
(S)—N-(3-cyano-4-(morpholin-2-yl)phenyl)-5-(trifluoromethyl)pyrazine-2-carboxamide
(R)—N-(3-fluoro-4-(morpholin-2-yl)phenyl)-5-methoxypyrazine-2-carboxamide
(S)—N-(3-fluoro-4-(morpholin-2-yl)phenyl)-5-methoxypyrazine-2-carboxamide
(S)-5-(cyclobutylmethoxy)-N-(3-fluoro-4-(morpholin-2-yl)phenyl)pyrazine-2-carboxamide
(S)-5-(cyclopropylmethoxy)-N-(3-fluoro-4-(morpholin-2-yl)phenyl)pyrazine-2-carboxamide
(S)-5-ethoxy-N-(3-fluoro-4-(morpholin-2-yl)phenyl)pyrazine-2-carboxamide
5-(3,3-difluoro-azetidin-1-yl)-pyrazine-2-carboxylic acid ((S)-3-fluoro-4-morpholin-2-yl-phenyl)-amide
(R)-5-cyclopropyl-N-(2-fluoro-4-(morpholin-2-yl)phenyl)pyrazine-2-carboxamide.
5-Cyclopropyl-pyrazine-2-carboxylic acid ((R)-3-fluoro-4-morpholin-2-yl-phenyl)-amide
5-cyclopropyl-pyrazine-2-carboxylic acid ((S)-3-fluoro-4-morpholin-2-yl-phenyl)-amide
(S)-5-cyclopropyl-N-(2-fluoro-4-(morpholin-2-yl)phenyl)pyrazine-2-carboxamide
(R)-5-cyclopropyl-N-(2-fluoro-4-(morpholin-2-yl)phenyl)pyrazine-2-carboxamide
(S)—N-(2-Fluoro-4-(morpholin-2-yl)phenyl)-5-(2,2,2-trifluoroethoxy)pyrazine-2-carboxamide
(S)—N-(3-Fluoro-4-(morpholin-2-yl)phenyl)-5-(2,2,2-trifluoroethoxy)pyrazine-2-carboxamide
(R)—N-(2-Fluoro-4-(morpholin-2-yl)phenyl)-5-(2,2,2-trifluoroethoxy)pyrazine-2-carboxamide or
(R)—N-(3-Fluoro-4-(morpholin-2-yl)phenyl)-5-(2,2,2-trifluoroethoxy)pyrazine-2-carboxamide.

One further embodiment of the invention are compounds of formula I, wherein R² is lower alkyl, lower alkoxy, lower alkyl substituted by halogen, lower alkoxy substituted by halogen, cycloalkyl, OCH₂-cycloalkyl or heterocycloalkyl, which is optionally substituted by halogen, and R¹ is hydrogen, for example the following compounds
(R)—N-(3-cyano-4-(morpholin-2-yl)phenyl)-6-(trifluoromethyl)pyrazine-2-carboxamide
(S)—N-(3-fluoro-4-(morpholin-2-yl)phenyl)-6-(trifluoromethyl)pyrazine-2-carboxamide
(R)—N-(3-fluoro-4-(morpholin-2-yl)phenyl)-6-(trifluoromethyl)pyrazine-2-carboxamide
(R)—N-(3-fluoro-4-(morpholin-2-yl)phenyl)-6-methoxypyrazine-2-carboxamide
(S)—N-(3-fluoro-4-(morpholin-2-yl)phenyl)-6-methoxypyrazine-2-carboxamide
6-Isopropyl-pyrazine-2-carboxylic acid ((S)-3-fluoro-4-morpholin-2-yl-phenyl)-amide
6-Isopropyl-pyrazine-2-carboxylic acid ((S)-2-fluoro-4-morpholin-2-yl-phenyl)-amide
(S)—N-(2-Fluoro-4-(morpholin-2-yl)phenyl)-6-(2,2,2-trifluoroethoxy)pyrazine-2-carboxamide
(S)—N-(3-Fluoro-4-(morpholin-2-yl)phenyl)-6-(2,2,2-trifluoroethoxy)pyrazine-2-carboxamide
(R)—N-(2-Fluoro-4-(morpholin-2-yl)phenyl)-6-(2,2,2-trifluoroethoxy)pyrazine-2-carboxamide or
(R)—N-(3-Fluoro-4-(morpholin-2-yl)phenyl)-6-(2,2,2-trifluoroethoxy)pyrazine-2-carboxamide.

One further embodiment of the invention are compounds of formula I, wherein R¹ and R² form together with the carbon atom to which they are attached a phenyl ring, which may be optionally substituted by lower alkyl, for example the following compounds
(S)—N-(3-fluoro-4-(morpholin-2-yl)phenyl)quinoxaline-2-carboxamide or
7-methyl-quinoxaline-2-carboxylic acid ((S)-3-fluoro-4-morpholin-2-yl-phenyl)-amide.

The preparation of compounds of formula I of the present invention may be carried out in sequential or convergent synthetic routes. Syntheses of the compounds of the invention are shown in the following schemes 1 & 2 and in the description of 20 specific examples. The skills required for carrying out the reaction and purification of the resulting products are known to those skilled in the art. The substituents and indices used in the following description of the processes have the significance given herein before unless indicated to the contrary.

In more detail, the compounds of formula I can be manufactured by the methods given below, by the methods given in the examples or by analogous methods. Appropriate reaction conditions for the individual reaction steps are known to a person skilled in the art. The reaction sequence is not limited to the one displayed in schemes 1 & 2, however, depending on the starting materials and their respective reactivity the sequence of reaction steps can be freely altered. Starting materials are either commercially available or can be prepared by methods analogous to the methods given below, by methods described in references cited in the description or in the examples, or by methods known in the art.

The present compounds of formula I and their pharmaceutically acceptable salts can be prepared by methods known in the art, for example, by processes described below, which process comprises

a) cleaving off the N-protecting group (PG) from compounds of formula

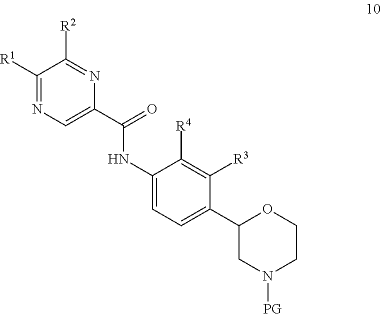

to a compound of formula

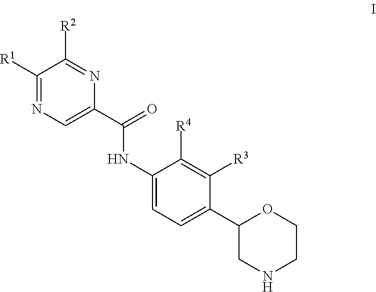

wherein PG is a N-protecting group selected from —C(O)O-tert-butyl and the other definitions are as described above, and,

if desired, converting the compounds obtained into pharmaceutically acceptable acid addition salts.

### General Procedure

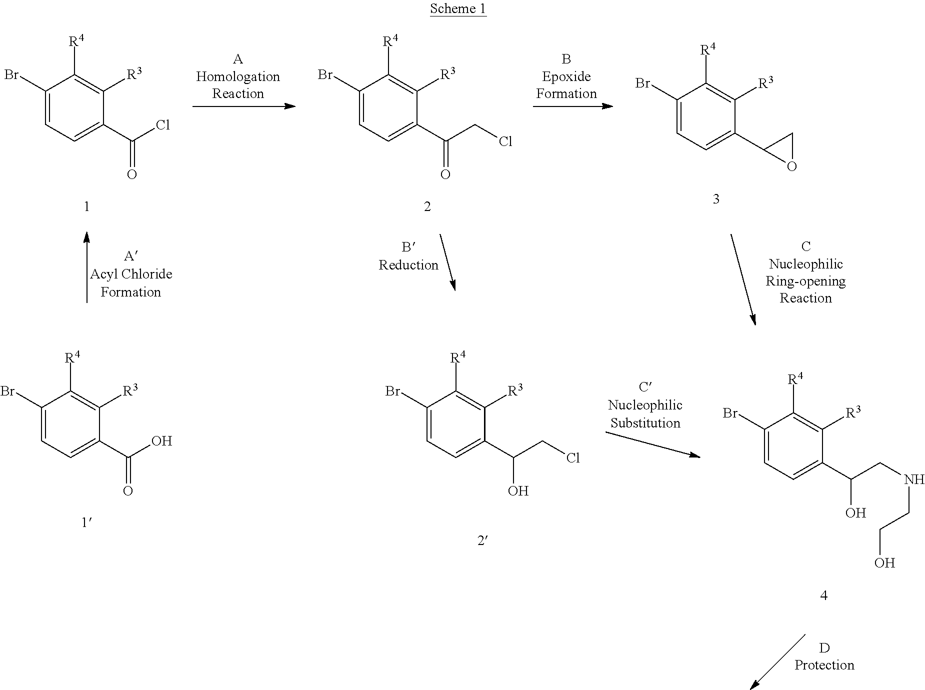

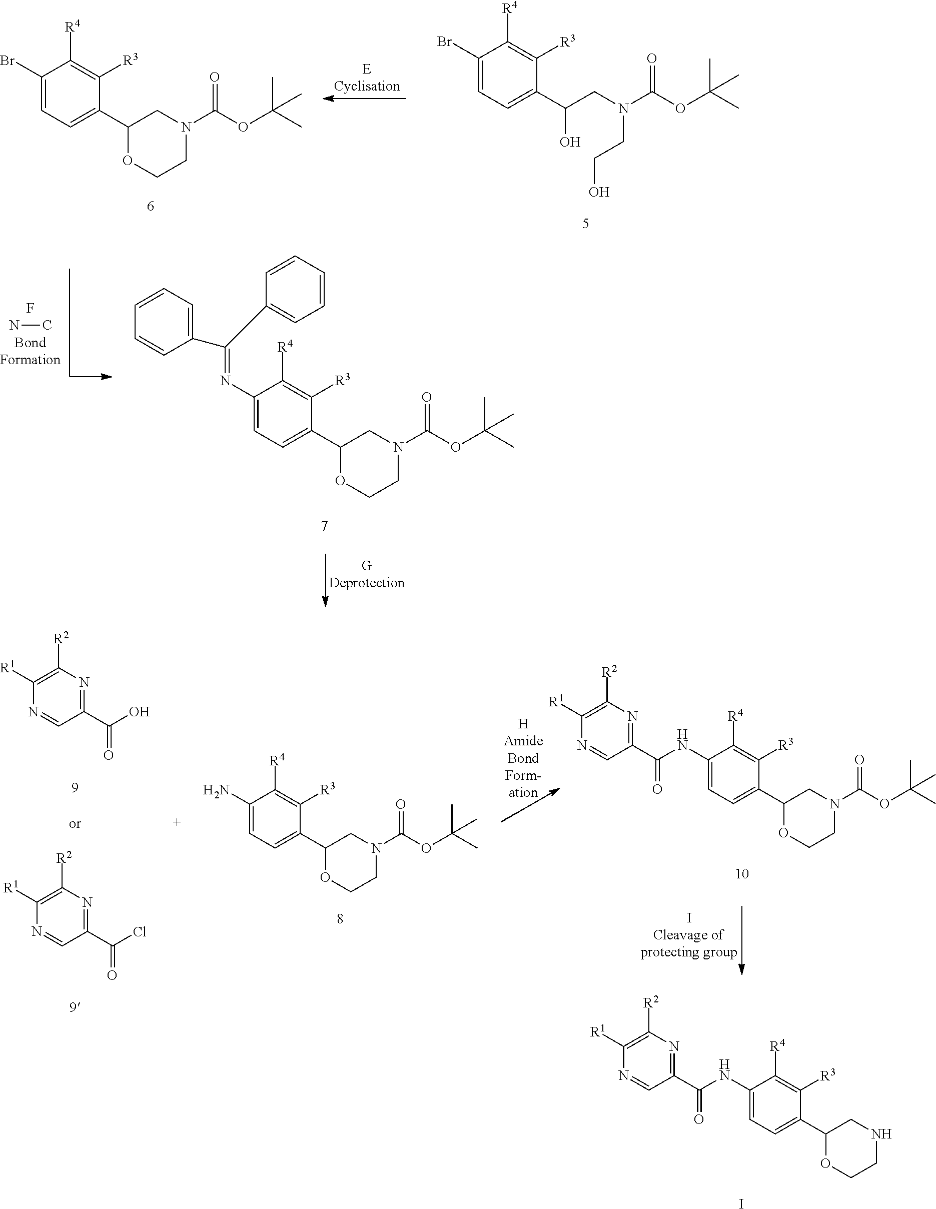

The substituents are as described above.

Step A:

Alpha-chloro ketone 2 can be obtained by a homologation reaction of acyl chloride 1 involving sequential treatment first with (trimethylsilyl)diazomethane and then treatment with concentrated hydrochloric acid. The reaction is carried out using a mixture of acetonitrile, THF and diethyl ether as solvent at temperatures between 0° C. and room temperature.

Preferred conditions are mixing of reactants at 0-5° C. followed by allowing to react for 30 minutes at room temperature for the first step, and mixing of reactants at 0-5° C. followed by allowing to react for 30 minutes at room temperature for the second step.

### Step A′:

In cases where the acyl chloride 1 is not commercially available, it may be prepared in situ from the corresponding carboxylic acid 1′, for instance by treatment with 1-chloro-N,N,2-trimethylpropenylamine [CAS 26189-59-3] in dichloromethane, followed by removal of the solvent in vacuo, according to the method of Ghosez and co-workers (J. Chem. Soc., Chem. Commun. 1979, 1180; Org. Synth. 1980, 59, 26-34).

### Step B:

Epoxide formation can be accomplished by a stepwise process involving reduction of alpha-chloro ketone 2 by treatment with a reducing agent such as NaBH₄ or LiBH₄ in a solvent such as MeOH, EtOH, THF, dioxane, followed by cyclisation of the ensuing alpha-chloro alcohol by treatment with a base such as sodium methoxide, sodium ethoxide, potassium tert-butoxide or caesium carbonate in the same solvent.

Preferred conditions are NaBH₄ in ethanol at 5° C. to room temperature for 1 hour followed by treatment with sodium methoxide at room temperature for 16 hours and then at 40° C. for 1 hour.

### Step C:

Nucleophilic ring-opening can be accomplished by treatment of epoxide 3 with 2-aminoethanol, optionally in the presence of an organic base such as triethylamine, N,N-diisopropylethylamine or N-methylmorpholine in a non-protic polar organic solvent such as ether, THF, dioxane or TBME.

Preferred conditions are using excess 2-aminoethanol as base in THF at room temperature for 16 hours.

### Step B′:

As an alternative to step B, the alpha-chloro ketone 2 may be treated with a reducing agent such as NaBH₄ or LiBH₄ in a solvent such as MeOH, EtOH, THF, dioxane, followed by isolation of the ensuing alpha-chloro alcohol 2′.

Preferred conditions are NaBH₄ in ethanol at 5° C. to room temperature for 2 hours.

### Step C′:

As an alternative to step C, the alpha-chloro alcohol 2′ prepared by step B′ may be treated with 2-aminoethanol, optionally in the presence of an organic base such as triethylamine, N,N-diisopropylethylamine or N-methylmorpholine in a non-protic polar organic solvent such as ether, THF, dioxane or TBME, preferably at elevated temperatures.

Preferred conditions are using excess 2-aminoethanol as base in THF at 90° C. for 16 hours.

### Step D:

Selective protection of the amino group of amino alcohol 4 can be effected by treatment with di-tert-butyl carbonate, optionally in the presence of an organic base such as triethylamine, N,N-diisopropylethylamine or N-methylmorpholine, in halogenated solvents such as dichloromethane or 1,2-dichloroethane or ethereal solvents such as diethyl ether, dioxane, THF or TBME.

Preferred conditions are dichloromethane in the absence of a base at room temperature for 16 hours.

### Step E:

Cyclisation can be accomplished by a stepwise process involving sulphonate ester formation by treatment of diol 5 with one equivalent of methanesulfonyl chloride in the presence of an organic base such as triethylamine, N,N-diisopropylethylamine or N-methylmorpholine in ethereal solvents such as diethyl ether, dioxane, THF or TBME, followed by cyclisation by treatment with a non-nucleophilic base such as potassium tert-butoxide or potassium 2-methyl-2-butoxide in ethereal solvents such as diethyl ether, dioxane, THF or TBME.

Preferred conditions for the first step are triethylamine in THF mixing the reactants at 0-5° C. and then allowing to react for 30 minutes at room temperature, then removal of the by-product triethylamine hydrochloride by filtration. Preferred conditions for the second step are potassium 2-methyl-2-butoxide in THF mixing the reactants at 0-5° C. and then allowing to react for 1 hour at room temperature.

As an alternative, cyclisation can be accomplished using Mitsunobu-like conditions involving treatment of diol 5 with a dialkyl diazodicarboxylate reagent such as diethyl diazodicarboxylate (DEAD) or diisopropyl diazodicarboxylate (DIAD) in the presence of a triarylphosphine such as triphenylphosphine in ethereal solvents such as diethyl ether, dioxane, THF or TBME.

Preferred conditions are DIAD and triphenylphosphine in TBME at room temperature for 16 hours.

### Step F:

C—N bond formation can be accomplished by treatment of 6 with benzophenone imine in the presence of a palladium or copper catalyst, a ligand and a base in solvents such as dioxane, DME, THF, toluene, DMF and DMSO at elevated temperatures, for instance using a palladium-catalysed Buchwald-Hartwig reaction.

Preferred conditions are catalytic tris(dibenzylidineacetone)dipalladium(0), catalytic (R)-(+)-2,2′-bis(diphenylphosphino)-1,1′-binaphthyl and sodium tert-butoxide in dioxane at 100° C. for 1 hour.

### Step G:

Removal of the nitrogen protecting group of 7 can be effected by hydrogenation with hydrogen under normal or elevated pressure or by transfer hydrogenation using ammonium formate or cyclohexadiene as hydrogen source with a catalyst such as PtO₂, Pd—C or Raney nickel in solvents such as MeOH, EtOH, H₂O, dioxane, THF, HOAc, EtOAc CH₂Cl₂, CHCl₃, DMF or mixtures thereof.

Preferred conditions are ammonium formate in the presence of palladium on charcoal in MeOH at 60° C. for 1 hour.

If desired, the racemic mixture of chiral amine 8 may be separated into its constituent enantiomers by using chiral HPLC.

### Step H:

Amide bond formation can be accomplished by a coupling reaction between amine 8 and a carboxylic acid compound 9 in the presence of a coupling reagent such as DCC, EDC, TBTU or HATU in the presence of an organic base such as triethylamine, N,N-diisopropylethylamine or N-methylmorpholine in halogenated solvents such as dichloromethane or 1,2-dichloroethane or ethereal solvents such as diethyl ether, dioxane, THF, DME or TBME.

Preferred conditions are TBTU with N-methylmorpholine in THF at 50-60° C. for 18-48 hours. Alternatively, amide bond formation can be accomplished by a coupling reaction between amine 8 and an acyl chloride compound 9′ in halogenated solvents such as dichloromethane or 1,2-dichloroethane or ethereal solvents such as diethyl ether, dioxane, THF, DME or TBME, in the presence of an organic base such as triethylamine or N,N-diisopropylethylamine.

Preferred conditions are triethylamine in THF at room temperature for 18 hours.

If desired, the acyl chloride compound 9′ may be prepared in situ from the corresponding carboxylic acid 9 by treatment with oxalyl chloride in halogenated solvents such as dichloromethane or 1,2-dichloroethane or ethereal solvents such as diethyl ether, dioxane, THF, DME or TBME in the presence of a catalyst such as DMF.

Preferred conditions are dichloroethane at room temperature for 1 hour.

Alternatively, the acyl chloride compound 9′ may be prepared in situ from the corresponding carboxylic acid 9 by treatment with 1-chloro-N,N,2-trimethylpropenylamine [CAS 26189-59-3] in dichloromethane, followed by removal of the solvent in vacuo, according to the method of Ghosez and co-workers (J. Chem. Soc., Chem. Commun. 1979, 1180; Org. Synth. 1980, 59, 26-34).

### Step I:

Removal of the BOC N-protecting group can be effected with mineral acids such as HCl, H₂SO₄ or H₃PO₄ or organic acids such as CF₃COOH, CHCl₂COOH, HOAc or p-toluenesulfonic acid in solvents such as CH₂Cl₂, CHCl₃, THF, MeOH, EtOH or H₂O at 0 to 80° C.

Preferred conditions are CF₃COOH in aqueous acetonitrile at 80° C. for 5 hours or 4 N HCl in dioxane at room temperature for 16 hours.

If desired, the racemic mixture of morpholine compounds I may be separated into its constituent enantiomers by using chiral HPLC.

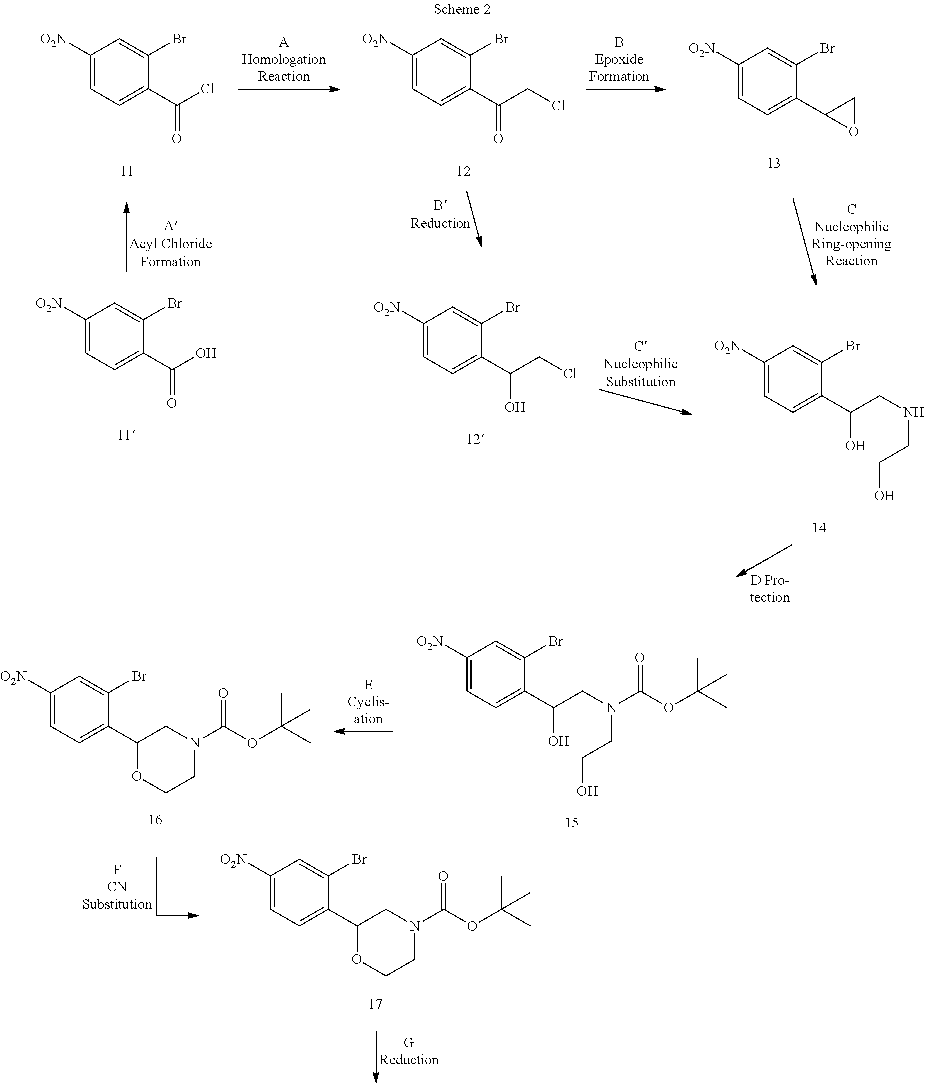

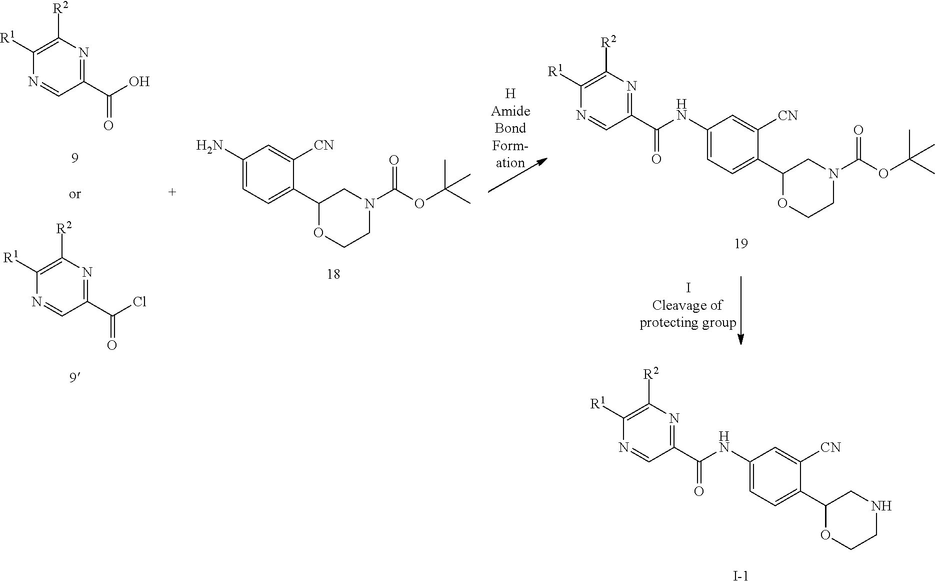

### Step A:

Alpha-chloro ketone 12 can be obtained by a homologation reaction of acyl chloride 11 involving sequential treatment first with (trimethylsilyl)diazomethane and then treatment with concentrated hydrochloric acid. The reaction is carried out using a mixture of acetonitrile, THF and diethyl ether as solvent at temperatures between 0° C. and room temperature.

Preferred conditions are mixing of reactants at 0-5° C. followed by allowing to react for 30 minutes at room temperature for the first step, and mixing of reactants at 0-5° C. followed by allowing to react for 30 minutes at room temperature for the second step.

### Step A′:

In cases where the acyl chloride 11 is not commercially available, it may be prepared in situ from the corresponding carboxylic acid 11′, for instance by treatment with 1-chloro-N,N,2-trimethylpropenylamine [CAS 26189-59-3] in dichloromethane, followed by removal of the solvent in vacuo, according to the method of Ghosez and co-workers (J. Chem. Soc., Chem. Commun. 1979, 1180; Org. Synth. 1980, 59, 26-34).

### Step B:

Epoxide formation can be accomplished by a stepwise process involving reduction of alpha-chloro ketone 12 by treatment with a reducing agent such as NaBH₄ or LiBH₄ in a solvent such as MeOH, EtOH, THF, dioxane, followed by cyclisation of the ensuing alpha-chloro alcohol by treatment with a base such as sodium methoxide, sodium ethoxide, potassium tert-butoxide or caesium carbonate in the same solvent.

Preferred conditions are NaBH₄ in ethanol at 5° C. to room temperature for 1 hour followed by treatment with sodium methoxide at room temperature for 16 hours and then at 40° C. for 1 hour.

### Step C:

Nucleophilic ring-opening can be accomplished by treatment of epoxide 13 with 2-aminoethanol, optionally in the presence of an organic base such as triethylamine, N,N-diisopropylethylamine or N-methylmorpholine in a non-protic polar organic solvent such as ether, THF, dioxane or TBME.

Preferred conditions are using excess 2-aminoethanol as base in THF at room temperature for 16 hours.

### Step B′:

As an alternative to step B, the alpha-chloro ketone 12 may be treated with a reducing agent such as NaBH₄ or LiBH₄ in a solvent such as MeOH, EtOH, THF, dioxane, followed by isolation of the ensuing alpha-chloro alcohol 12′.

Preferred conditions are NaBH₄ in ethanol at 5° C. to room temperature for 2 hours.

### Step C′:

As an alternative to step C, the alpha-chloro alcohol 12′ prepared by step B′ may be treated with 2-aminoethanol, optionally in the presence of an organic base such as triethylamine, N,N-diisopropylethylamine or N-methylmorpholine in a non-protic polar organic solvent such as ether, THF, dioxane or TBME, preferably at elevated temperatures.

Preferred conditions are using excess 2-aminoethanol as base in THF at 90° C. for 16 hours.

### Step D:

Selective protection of the amino group of amino alcohol 14 can be effected by treatment with di-tert-butyl carbonate, optionally in the presence of an organic base such as triethylamine, N,N-diisopropylethylamine or N-methylmorpholine, in halogenated solvents such as dichloromethane or 1,2-dichloroethane or ethereal solvents such as diethyl ether, dioxane, THF or TBME.

Preferred conditions are dichloromethane in the absence of a base at room temperature for 16 hours.

### Step E:

Cyclisation can be accomplished by a stepwise process involving sulphonate ester formation by treatment of diol 15 with one equivalent of methanesulfonyl chloride in the presence of an organic base such as triethylamine, N,N-diisopropylethylamine or N-methylmorpholine in ethereal solvents such as diethyl ether, dioxane, THF or TBME, followed by cyclisation by treatment with a non-nucleophilic base such as potassium tert-butoxide or potassium 2-methyl-2-butoxide in ethereal solvents such as diethyl ether, dioxane, THF or TBME.

Preferred conditions for the first step are triethylamine in THF mixing the reactants at 0-5° C. and then allowing to react for 30 minutes at room temperature, then removal of the by-product triethylamine hydrochloride by filtration. Preferred conditions for the second step are potassium 2-methyl-2-butoxide in THF mixing the reactants at 0-5° C. and then allowing to react for 1 hour at room temperature.

As an alternative, cyclisation can be accomplished using Mitsunobu-like conditions involving treatment of diol 15 with a dialkyl diazodicarboxylate reagent such as diethyl diazodicarboxylate (DEAD) or diisopropyl diazodicarboxylate (DIAD) in the presence of a triarylphosphine such as triphenylphosphine in ethereal solvents such as diethyl ether, dioxane, THF or TBME.

Preferred conditions are DIAD and triphenylphosphine in TBME at room temperature for 16 hours.

### Step F:

Aromatic nitrile compound 17 can be prepared by reaction of aromatic bromine compound 16 with metal cyanide salts such as potassium cyanide, sodium cyanide, zinc cyanide or copper(I) cyanide, optionally in the presence of a palladium catalyst.

The reaction is carried out in non-protic polar organic solvents such as DMF or NMP at elevated temperatures.

Preferred conditions are Zn(CN)₂ with tetrakis(triphenylphosphine)palladium(0) in DMF at 160° C. for 30 mins under microwave irradiation in a sealed tube.

### Step G:

Reduction of the nitro group of 17 can be effected by hydrogenation with hydrogen under normal or elevated pressure in the presence of a catalyst such as PtO₂, Pd—C or Raney nickel in solvents such as MeOH, EtOH, H₂O, dioxane, THF, HOAc, EtOAc, DMF or mixtures thereof.

Preferred conditions are palladium on charcoal in EtOH and EtOAc at room temperature and 1 atm H₂ for 72 hours.

If desired, the racemic mixture of chiral amine 18 may be separated into its constituent enantiomers by using chiral HPLC.

### Step H:

Amide bond formation can be accomplished by a coupling reaction between amine 18 and a carboxylic acid compound 9 in the presence of a coupling reagent such as DCC, EDC, TBTU or HATU in the presence of an organic base such as triethylamine, N,N-diisopropylethylamine or N-methylmorpholine in halogenated solvents such as dichloromethane or 1,2-dichloroethane or ethereal solvents such as diethyl ether, dioxane, THF, DME or TBME. Preferred conditions are TBTU with N-methylmorpholine in THF at 50-60° C. for 18-48 hours. Alternatively, amide bond formation can be accomplished by a coupling reaction between amine 18 and an acyl chloride compound 9′ in halogenated solvents such as dichloromethane or 1,2-dichloroethane or ethereal solvents such as diethyl ether, dioxane, THF, DME or TBME, in the presence of an organic base such as triethylamine or N,N-diisopropylethylamine.

Preferred conditions are triethylamine in THF at room temperature for 18 hours.

If desired, the acyl chloride compound 9′ may be prepared in situ from the corresponding carboxylic acid 9 by treatment with oxalyl chloride in halogenated solvents such as dichloromethane or 1,2-dichloroethane or ethereal solvents such as diethyl ether, dioxane, THF, DME or TBME in the presence of a catalyst such as DMF.

Preferred conditions are dichloroethane at room temperature for 1 hour.

Alternatively, the acyl chloride compound 9′ may be prepared in situ from the corresponding carboxylic acid 9 by treatment with 1-chloro-N,N,2-trimethylpropenylamine [CAS 26189-59-3] in dichloromethane, followed by removal of the solvent in vacuo, according to the method of Ghosez and co-workers (J. Chem. Soc., Chem. Commun. 1979, 1180; Org. Synth. 1980, 59, 26-34).

### Step I:

Removal of the BOC N-protecting group can be effected with mineral acids such as HCl, H₂SO₄ or H₃PO₄ or organic acids such as CF₃COOH, CHCl₂COOH, HOAc or p-toluenesulfonic acid in solvents such as CH₂Cl₂, CHCl₃, THF, MeOH, EtOH or H₂O at 0 to 80° C.

Preferred conditions are CF₃COOH in aqueous acetonitrile at 80° C. for 5 hours or 4 N HCl in dioxane at room temperature for 16 hours.

If desired, the racemic mixture of morpholine compounds I-1 may be separated into its constituent enantiomers by using chiral HPLC.

### Isolation and Purification of the Compounds

Isolation and purification of the compounds and intermediates described herein can be effected, if desired, by any suitable separation or purification procedure such as, for example, filtration, extraction, crystallization, column chromatography, thin-layer chromatography, thick-layer chromatography, preparative low or high-pressure liquid chromatography or a combination of these procedures. Specific illustrations of suitable separation and isolation procedures can be had by reference to the preparations and examples herein below. However, other equivalent separation or isolation procedures could, of course, also be used. Racemic mixtures of chiral compounds of formula I can be separated using chiral HPLC. Racemic mixtures of chiral synthetic intermediates may also be separated using chiral HPLC.

### Salts of Compounds of Formula I

The compounds of formula I are basic and may be converted to a corresponding acid addition salt. The conversion is accomplished by treatment with at least a stoichiometric amount of an appropriate acid, such as hydrochloric acid, hydrobromic acid, sulfuric acid, nitric acid, phosphoric acid and the like, and organic acids such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid and the like. Typically, the free base is dissolved in an inert organic solvent such as diethyl ether, ethyl acetate, chloroform, ethanol or methanol and the like, and the acid added in a similar solvent. The temperature is maintained between 0° C. and 50° C. The resulting salt precipitates spontaneously or may be brought out of solution with a less polar solvent.

### EXAMPLE 1

### (R)—N-(3-Fluoro-4-(morpholin-2-yl)phenyl)-5-(trifluoromethyl)pyrazine-2-carboxamide hydrochloride

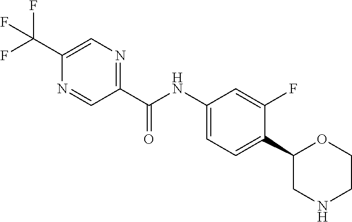 [SMILES: O=C(Nc1ccc([C@@H]2CNCCO2)c(F)c1)c1cnc(C(F)(F)F)cn1]

### a) 1-(4-Bromo-2-fluoro-phenyl)-2-chloro-ethanone

To a stirred solution of 4-bromo-2-fluorobenzoic acid (10.0 g, CAS 112704-79-7) in dichloromethane (60 ml) was added 1-chloro-N,N,2-trimethylpropenylamine (6.95 ml) and the reaction mixture was stirred at RT over 15 minutes. The reaction mixture became a yellow solution. The solvent was evaporated and the residue was diluted in THF (100 ml) and acetonitrile (100 ml). The resulting solution was cooled to 0-5° C. and (trimethylsilyl)diazomethane (27.4 ml, 2 M solution in hexane) was added dropwise. The reaction mixture was stirred at room temperature for 30 min (gas evolution). TLC analysis showed the reaction was complete. Hydrochloric acid (7.61 ml, 37% aq.) was then added dropwise at 0-5° C. over 10 minutes and the reaction mixture was then stirred at room temperature for a further 1 hour. The reaction mixture was poured into EtOAc and extracted sequentially with aq. Na₂CO₃ solution, water and saturated brine. The organic layer was then dried over MgSO₄ and concentrated in vacuo. The crude material was triturated four times in dichloromethane and the resulting solid was collected by filtration to afford 1-(4-bromo-2-fluoro-phenyl)-2-chloro-ethanone (12.22 g) as a yellow solid which was used in the next step without further purification.

### b) (RS)-1-(4-bromo-2-fluoro-phenyl)-2-chloroethanol

To a stirred solution of 1-(4-bromo-2-fluoro-phenyl)-2-chloro-ethanone (12.22 g) in ethanol (200 ml) at 0° C. was added portionwise over 5 min NaBH₄ (2.04 g). The reaction mixture was then stirred at room temperature for 2 hours to afford an orange solution. TLC analysis showed the reaction was complete. The reaction mixture was then poured into water and extracted twice with EtOAc. The combined organic layers were washed with saturated brine, then dried over MgSO₄ and concentrated in vacuo to afford (RS)-1-(4-bromo-2-fluoro-phenyl)-2-chloroethanol (11.48 g) as a yellow oil which was used in the next step without further purification.

### c) (RS)-1-(4-Bromo-2-fluoro-phenyl)-2-(2-hydroxy-ethylamino)-ethanol

To a stirred solution of (RS)-1-(4-bromo-2-fluoro-phenyl)-2-chloroethanol (11.48 g) in THF (28 ml) was added 2-aminoethanol (27.6 ml) and the mixture was stirred at 90° C. overnight. The reaction mixture was then poured into brine and extracted twice with EtOAc. The combined organic layers was dried over MgSO₄ and concentrated in vacuo to afford (RS)-1-(4-bromo-2-fluoro-phenyl)-2-(2-hydroxy-ethylamino)-ethanol (12.49 g) as a yellow viscous oil which was used in the next step without further purification. MS (ISP): 280.0 ([{⁸¹Br}M+H]⁺), 278.0 ([{⁷⁹Br}M+H]⁺).

### d) (RS)-[2-(4-Bromo-2-fluoro-phenyl)-2-hydroxy-ethyl]-(2-hydroxy-ethyl)-carbamic acid tert-butyl ester

To a stirred solution of (RS)-1-(4-bromo-2-fluoro-phenyl)-2-(2-hydroxy-ethylamino)-ethanol (12.49 g) in THF (125 ml) was added Boc₂O (10.8 g) and the mixture was stirred at room temperature for 4 hours. The reaction mixture was then concentrated in vacuo and the residue was partitioned between aq. NaOH and EtOAc. The layers were separated and the organic phase was dried over MgSO₄ and concentrated in vacuo. The residue was purified by flash column chromatography (silica gel; gradient: 20% to 60% EtOAc in heptane) to afford (RS)-[2-(4-bromo-2-fluoro-phenyl)-2-hydroxy-ethyl]-(2-hydroxy-ethyl)-carbamic acid tert-butyl ester (9.83 g, 58% over 4 steps) as a light yellow oil. MS (ISP): 380.1 ([{⁸¹Br}M+H]⁺), 378.1 ([{⁷⁹Br}M+H]⁺).

### e) (RS)-2-(4-Bromo-2-fluoro-phenyl)-morpholine-4-carboxylic acid tert-butyl ester

To a stirred solution of (RS)-[2-(4-bromo-2-fluoro-phenyl)-2-hydroxy-ethyl]-(2-hydroxy-ethyl)-carbamic acid tert-butyl ester (7.39 g) and triphenylphosphine (6.15 g) in TBME (33 ml) was added DIAD (4.85 ml) under ice-bath cooling (exotherm). The yellow solution was stirred at RT overnight. The reaction mixture became a yellow suspension. The solvent was evaporated, TBME was then added and the solid was filtered off. The filtrate was evaporated and the crude material was purified by flash column chromatography (silica gel; gradient: 5% to 40% EtOAc in heptane) to afford (RS)-2-(4-bromo-2-fluoro-phenyl)-morpholine-4-carboxylic acid tert-butyl ester (5.43 g, 77%) as a yellow oil. MS (EI): 361 ([{⁸¹Br}M⁺), 359 ([{⁷⁹Br}M⁺), 305 ([{⁸¹Br}M-C₄H₈]⁺), 303 ([{⁷⁹Br}M-C₄H₈]⁺), 260 ([{⁸¹Br}M-C₄H₈—CO₂H]⁺), 258 ([{⁷⁹Br}M- C₄H₈—CO₂H]⁺).

### f) (RS)-2-[4-(diphenylmethyleneamino)-2-fluoro-phenyl]-morpholine-4-carboxylic acid tert-butyl ester

To a stirred solution of (RS)-2-(4-bromo-2-fluoro-phenyl)-morpholine-4-carboxylic acid tert-butyl ester (6.21 g) and benzophenone imine (3.35 ml) in toluene (43 ml) was added sodium tert-butoxide (2.7 g). The reaction mixture was purged with argon for 10 min. (R)-(+)-2,2′-bis(diphenylphosphino)-1,1′-binaphthyl (1.11 g) and tris(dibenzylideneacetone)dipalladium(0) (488 mg) were added and the reaction mixture was heated to 90° C. and stirred for 1 h. The reaction mixture was poured into water and extracted twice with EtOAc. The organic layers were dried over MgSO₄ and concentrated in vacuo. The residue was purified by flash column chromatography (silica gel; gradient: 0% to 15% EtOAc in hexanes) to afford (RS)-2-[4-(diphenylmethyleneamino)-2-fluoro-phenyl]-morpholine-4-carboxylic acid tert-butyl ester (8.38 g, quant.) as an orange foam. MS (ISP): 461.2 ([M+H]⁺).

### g) (RS)-2-(4-Amino-2-fluoro-phenyl)-morpholine-4-carboxylic acid tert-butyl ester

To a stirred solution of (RS)-2-[4-(diphenylmethyleneamino)-2-fluoro-phenyl]-morpholine-4-carboxylic acid tert-butyl ester (8.225 g) in methanol (85 ml) were added sodium acetate (4.4 g) and hydroxylamine hydrochloride (2.73 g) and the reaction mixture was stirred at 60° C. overnight. The reaction mixture was then cooled to room temperature and partitioned between 1 M aq. NaOH and EtOAc. The layers were separated and the organic layer was dried over MgSO₄ and concentrated in vacuo. The residue was purified by flash column chromatography (silica gel; gradient: 5% to 60% EtOAc in heptane) to afford (RS)-2-(4-amino-2-fluoro-phenyl)-morpholine-4-carboxylic acid tert-butyl ester (5.13 g, 97%) as a white foam. MS (EI): 296 (M⁺).

### h) (+)-(R)-2-(4-Amino-2-fluoro-phenyl)-morpholine-4-carboxylic acid tert-butyl ester & (−)-(S)-2-(4-Amino-2-fluoro-phenyl)-morpholine-4-carboxylic acid tert-butyl ester

The enantiomers of (RS)-2-(4-amino-2-fluoro-phenyl)-morpholine-4-carboxylic acid tert-butyl ester (5.13 g) were separated using chiral HPLC (column: Chiralpak AD, 5×50 cm; eluent: 15% isopropanol/heptane; pressure: 18 bar; flow rate: 35 ml/min) affording:

(+)-(R)-2-(4-Amino-2-fluoro-phenyl)-morpholine-4-carboxylic acid tert-butyl ester (1.78 g, off-white solid), Retention time=83 min
(−)-(S)-2-(4-Amino-2-fluoro-phenyl)-morpholine-4-carboxylic acid tert-butyl ester (2.07 g, light yellow solid), Retention time=96 min

### i) (R)-2-(2-Fluoro-4-(5-(trifluoromethyl)pyrazine-2-carboxamido)phenyl)morpholine-4-carboxylic acid tert-butyl ester

To a stirred suspension of 5-(trifluoromethyl)pyrazine-2-carboxylic acid (157 mg, CAS 1060814-50-7) in dichloromethane (2.5 ml) was added dropwise 1-chloro-N,N,2-trimethylpropenylamine (113 μl) and the mixture was stirred at RT for 10 minutes during which time it became a colourless solution. A solution containing ethyldiisopropylamine (308 μl) and (R)-2-(4-amino-2-fluorophenyl)morpholine-4-carboxylic acid tert-butyl ester (220 mg) in dichloromethane (2.5 ml) was then added (the reaction mixture became light yellow) and the reaction mixture was stirred at RT for 60 minutes. TLC analysis showed the reaction was complete. The reaction mixture was partitioned between dichloromethane and aqueous citric acid. The phases were separated and the organic phase was dried over MgSO₄, filtered and concentrated in vacuo. The crude material was purified by flash chromatography (silica gel, gradient: 5% to 50% EtOAc in heptane) to afford (R)-2-(2-fluoro-4-(5-(trifluoromethyl)pyrazine-2-carboxamido)phenyl)morpholine-4-carboxylic acid tert-butyl ester (145 mg, 42%) as a white solid. MS (ISP): 469.6 ([M−H]⁻).

### j) 5-Trifluoromethyl-pyrazine-2-carboxylic acid ((R)-3-fluoro-4-morpholin-2-yl-phenyl)-amide hydrochloride

To a stirred solution of (R)-2-(2-fluoro-4-(5-(trifluoromethyl)pyrazine-2-carboxamido)phenyl)morpholine-4-carboxylic acid tert-butyl ester (159 mg) in dioxane (0.5 ml) was added dropwise a solution of 4 M HCl in dioxane (1.26 ml). The reaction mixture was stirred at RT overnight, during which a solid precipitated. The solvent was evaporated and the residue was dried under high vacuum to afford 5-trifluoromethyl-pyrazine-2-carboxylic acid ((R)-3-fluoro-4-morpholin-2-yl-phenyl)-amide hydrochloride (66 mg, 48%) as a white solid. MS (ISP): 371.4 ([M+H]⁺).

### Example 2

### (R)—N-(3-Cyano-4-(morpholin-2-yl)phenyl)-5-(trifluoromethyl)pyrazine-2-carboxamide hydrochloride

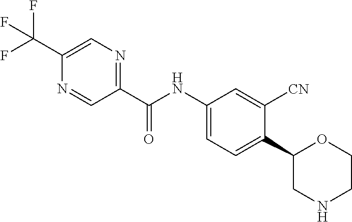 [SMILES: N#Cc1cc(NC(=O)c2cnc(C(F)(F)F)cn2)ccc1[C@@H]1CNCCO1]

### a) 1-(2-Bromo-4-nitrophenyl)-2-chloroethanone

To a stirred suspension of 2-bromo-4-nitrobenzoic acid (5.00 g, CAS 16426-64-5) in dichloromethane (20 ml) was added 1-chloro-N,N,2-trimethylpropenylamine (3.09 ml) and the reaction mixture was stirred at RT over 15 minutes. The reaction mixture became a yellow solution. The solvent was evaporated and the residue was diluted in THF (50 ml) and acetonitrile (50 ml). The resulting solution was cooled to 0-5° C. and (trimethylsilyl)diazomethane (12.2 ml, 2 M solution in diethyl ether) was added dropwise. The reaction mixture was stirred at room temperature for 30 min (gas evolution). TLC analysis showed the reaction was complete. Hydrochloric acid (3.39 ml, 37% aq.) was then added dropwise at 0-5° C. over 10 minutes and the reaction mixture was then stirred at room temperature for a further 1 hour. The reaction mixture was partitioned between EtOAc and 2 M aq. Na₂CO₃ solution. The phases were separated and the organic layer was dried over MgSO₄ and concentrated in vacuo to afford 1-(2-bromo-4-nitrophenyl)-2-chloroethanone (5.82 g) as a brown solid which was used in the next step without further purification. MS (ISP): 278.1 ([{⁸¹Br}M−H]⁻), 276.1 ([{⁷⁹Br}M−H]⁻).

### b) (RS)-1-(2-bromo-4-nitrophenyl)-2-chloro ethanol

To a stirred solution of 1-(2-bromo-4-nitrophenyl)-2-chloroethanone (5.80 g) in ethanol (125 ml) at 0° C. was added portionwise over 5 min NaBH₄ (867 mg). The reaction mixture was then stirred at 0° C. for 30 minutes to afford an orange solution. TLC analysis showed the reaction was complete. The reaction mixture was then poured into water and extracted twice with EtOAc. The combined organic layers were washed with saturated brine, then dried over MgSO₄ and concentrated in vacuo to afford (RS)-1-(2-bromo-4-nitrophenyl)-2-chloroethanol (5.65 g) as a brown solid which was used in the next step without further purification. MS (ISP): 280.1 ([{⁸¹Br}M−H]⁻), 278.1 ([{⁷⁹Br}M−H]⁻).

### c) (RS)-1-(2-Bromo-4-nitrophenyl)-2-(2-hydroxyethylamino)ethanol

To a stirred solution of (RS)-1-(2-bromo-4-nitrophenyl)-2-chloroethanol (5.65 g) in THF (12 ml) was added 2-aminoethanol (12.3 ml) and the mixture was stirred at 90° C. overnight. TLC analysis showed the reaction was complete. The reaction mixture was then poured into brine and extracted twice with EtOAc. The combined organic layers was dried over MgSO₄ and concentrated in vacuo to afford (RS)-1-(2-bromo-4-nitrophenyl)-2-(2-hydroxyethylamino)ethanol (5.88 g) as a brown gum which was used in the next step without further purification. MS (ISP): 307.3 ([{⁸¹Br}M+H]⁺), 305.3 ([{⁷⁹Br}M+H]⁺).

### d) (RS)-2-(2-bromo-4-nitrophenyl)-2-hydroxyethyl(2-hydroxyethyl)carbamic acid tert-butyl ester

To a stirred solution of (RS)-1-(2-bromo-4-nitrophenyl)-2-(2-hydroxyethylamino)ethanol (5.87 g) in THF (60 ml) was added Boc₂O (4.62 g) and the mixture was stirred at room temperature for 2.5 hours. TLC analysis showed the reaction was complete. The reaction mixture was then concentrated in vacuo and the residue was partitioned between aq. NaOH and EtOAc. The layers were separated and the organic phase was dried over MgSO₄ and concentrated in vacuo. The residue was purified by flash column chromatography (silica gel; gradient: 20% to 100% EtOAc in heptane) to afford (RS)-2-(2-bromo-4-nitrophenyl)-2-hydroxyethyl(2-hydroxyethyl)carbamic acid tert-butyl ester (4.00 g, 51% over 4 steps) as a yellow gum. MS (ISP): 351.2 ([{⁸¹Br}M+H—C₄H₈]⁺), 349.2 ([{⁷⁹Br}M+H—C₄H₈]⁺).

### e) (RS)-2-(2-Bromo-4-nitrophenyl)morpholine-4-carboxylic acid tert-butyl ester

To a stirred solution of (RS)-2-(2-bromo-4-nitrophenyl)-2-hydroxyethyl(2-hydroxyethyl)carbamic acid tert-butyl ester (2.79 g) and triphenylphosphine (2.17 g) in TBME (13 ml) was added DIAD (1.71 ml) under ice-bath cooling (exotherm). The yellow solution was stirred at RT for 2 hours. The reaction mixture became a yellow suspension. The solvent was evaporated. The crude material was purified by flash column chromatography (silica gel; gradient: 5% to 40% EtOAc in heptane) to afford (RS)-2-(2-bromo-4-nitrophenyl)morpholine-4-carboxylic acid tert-butyl ester (1.78 g, 67%) as a light yellow solid. MS (ISP): 333.2 ([{⁸¹Br}M+H—C₄H₈]⁺), 331.2 ([{⁷⁹Br}M+H—C₄H₈]⁺).

### f) (RS)-2-(2-Cyano-4-nitrophenyl)morpholine-4-carboxylic acid tert-butyl ester

To a stirred solution of (RS)-2-(2-bromo-4-nitrophenyl)morpholine-4-carboxylic acid tert-butyl ester (2.12 g) in DMF (36 ml) in a microwave vial were added zinc cyanide (770 mg) and tetrakis(triphenylphosphine)palladium(0) (632 mg). The reaction vial was capped and the mixture was stirred under microwave irradiation at 160° C. over 30 minutes. TLC analysis showed the reaction was complete. The reaction mixture was partitioned between EtOAc and water, then the phases were separated and the organic phase was dried over MgSO₄, filtered, and concentrated in vacuo. The crude material was purified by flash chromatography (silica gel, eluent: 5% to 40% EtOAc in heptane) to afford (RS)-2-(2-cyano-4-nitrophenyl)morpholine-4-carboxylic acid tert-butyl ester (1.08 g, 60%) as a yellow solid. MS (ISP): 332.4 ([M−H]⁻).

### g) (RS)-2-(4-Amino-2-cyano-phenyl)morpholine-4-carboxylic acid tert-butyl ester

To a stirred solution of (RS)-2-(2-cyano-4-nitrophenyl)morpholine-4-carboxylic acid tert-butyl ester (1.00 g) in ethanol (10 ml) and ethyl acetate (15 ml) was added Pd on charcoal (100 mg). The reaction mixture was stirred under a hydrogen atmosphere for 72 hours. TLC analysis showed the reaction was complete. The catalyst was removed by filtration over dicalite. The mother liquor was concentrated in vacuo to afford (RS)-2-(4-amino-2-cyano-phenyl)morpholine-4-carboxylic acid tert-butyl ester (0.91 g, quant.) as a light grey foam. MS (ISP): 304.4 ([M+H]⁺), 248.4 ([M+H—C₄H₈]⁺), 204.4 ([M+H—C₄H₈—CO₂]⁺).

### h) (−)-(R)-2-(4-Amino-2-cyano-phenyl)morpholine-4-carboxylic acid tert-butyl ester & (+)-(S)-2-(4-Amino-2-cyano-phenyl)morpholine-4-carboxylic acid tert-butyl ester

The enantiomers of (RS)-2-(4-amino-2-cyano-phenyl)morpholine-4-carboxylic acid tert-butyl ester (1.00 g) were separated using chiral HPLC (column: Chiralpak AD, 5×50 cm; eluent: 20% isopropanol/heptane; pressure: 15 bar; flow rate: 35 ml/min) affording:

(−)-(R)-2-(4-amino-2-cyano-phenyl)morpholine-4-carboxylic acid tert-butyl ester (421 mg, white foam), Retention time=54 min
(+)-(S)-2-(4-amino-2-cyano-phenyl)morpholine-4-carboxylic acid tert-butyl ester (405 mg, white foam), Retention time=81 min

### i) (R)-2-(2-Cyano-4-(5-(trifluoromethyl)pyrazine-2-carboxamido)phenyl)morpholine-4-carboxylic acid tert-butyl ester

To a stirred suspension of 5-(trifluoromethyl)pyrazine-2-carboxylic acid (40 mg, CAS 1060814-50-7) in dichloromethane (800 μl) was added dropwise 1-chloro-N,N,2-trimethylpropenylamine (32 μl) and the mixture was stirred at RT for 15 minutes during which time it became a colourless solution. A solution containing ethyldiisopropylamine (78 μl) and (R)-2-(4-amino-2-cyano-phenyl)morpholine-4-carboxylic acid tert-butyl ester (57 mg) in DMF (800 μl) was then added (the reaction mixture became light yellow) and the reaction mixture was stirred at RT for 30 minutes. TLC analysis showed the reaction was complete. The reaction mixture was partitioned between ethyl acetate and aqueous citric acid. The phases were separated and the organic phase was dried over MgSO₄, filtered and concentrated in vacuo. The crude material was purified by flash chromatography (silica gel, gradient: 5% to 50% EtOAc in heptane) to afford (R)-2-(2-cyano-4-(5-(trifluoromethyl)pyrazine-2-carboxamido)phenyl)morpholine-4-carboxylic acid tert-butyl ester (77 mg, 86%) as a white solid. MS (ISP): 476.3 ([M−H]⁻).

### j) (R)—N-(3-Cyano-4-(morpholin-2-yl)phenyl)-5-(trifluoromethyl)pyrazine-2-carboxamide hydrochloride

To a stirred solution of (R)-2-(2-cyano-4-(5-(trifluoromethyl)pyrazine-2-carboxamido)phenyl)morpholine-4-carboxylic acid tert-butyl ester (90 mg) in dioxane (0.3 ml) was added dropwise a solution of 4 M HCl in dioxane (705 μl). The reaction mixture was stirred at RT overnight, during which time a solid precipitated. The solvent was evaporated and the residue was triturated in ethanol and diethyl ether and then dried under high vacuum to afford (R)—N-(3-cyano-4-(morpholin-2-yl)phenyl)-5-(trifluoromethyl)pyrazine-2-carboxamide hydrochloride (43 mg, 55%) as a white solid. MS (ISP): 378.4 ([M+H]⁺).

### Example 3

### (S)—N-(3-Cyano-4-(morpholin-2-yl)phenyl)-5-(trifluoromethyl)pyrazine-2-carboxamide hydrochloride

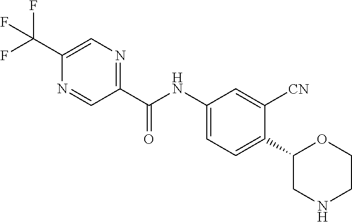 [SMILES: N#Cc1cc(NC(=O)c2cnc(C(F)(F)F)cn2)ccc1[C@H]1CNCCO1]

The title compound was obtained in analogy to example 2 using (S)-2-(4-amino-2-cyano-phenyl)morpholine-4-carboxylic acid tert-butyl ester in place of (R)-2-(4-amino-2-cyano-phenyl)morpholine-4-carboxylic acid tert-butyl ester in step (i). White solid. MS (ISP): 378.3 ([M+H]⁺).

### Example 4

### (R)—N-(3-Cyano-4-(morpholin-2-yl)phenyl)-6-(trifluoromethyl)pyrazine-2-carboxamide hydrochloride

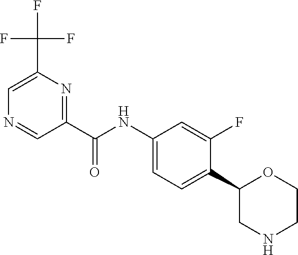 [SMILES: O=C(Nc1ccc([C@@H]2CNCCO2)c(F)c1)c1cncc(C(F)(F)F)n1]

The title compound was obtained in analogy to example 2 using 6-(trifluoromethyl)pyrazine-2-carboxylic acid (CAS 1060812-74-9) in place of 5-(trifluoromethyl)pyrazine-2-carboxylic acid in step (i). Off-white solid. MS (ISP): 376.3 ([M−H]⁻).

### Example 5

### (S)—N-(3-Fluoro-4-(morpholin-2-yl)phenyl)-6-(trifluoromethyl)pyrazine-2-carboxamide hydrochloride

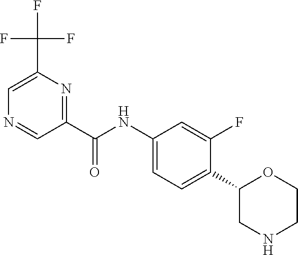 [SMILES: O=C(Nc1ccc([C@H]2CNCCO2)c(F)c1)c1cncc(C(F)(F)F)n1]

The title compound was obtained in analogy to example 1 using (−)-(S)-2-(4-amino-2-fluoro-phenyl)-morpholine-4-carboxylic acid tert-butyl ester in place of (+)-(R)-2-(4-amino-2-fluoro-phenyl)-morpholine-4-carboxylic acid tert-butyl ester and 6-(trifluoromethyl)pyrazine-2-carboxylic acid (CAS 1060812-74-9) in place of 5-(trifluoromethyl)pyrazine-2-carboxylic acid in step (i). Off-white solid. MS (ISP): 371.4 ([M+H]⁺).

### Example 6

### (R)—N-(3-Fluoro-4-(morpholin-2-yl)phenyl)-6-(trifluoromethyl)pyrazine-2-carboxamide hydrochloride

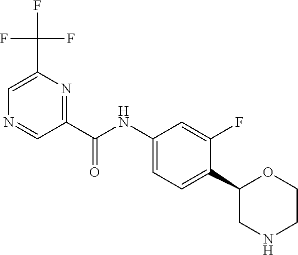 [SMILES: O=C(Nc1ccc([C@@H]2CNCCO2)c(F)c1)c1cncc(C(F)(F)F)n1]

The title compound was obtained in analogy to example 1 using 6-(trifluoromethyl)pyrazine-2-carboxylic acid (CAS 1060812-74-9) in place of 5-(trifluoromethyl)pyrazine-2-carboxylic acid in step (i). White solid. MS (ISP): 371.4 ([M+H]⁻).

### Example 7

### (R)—N-(3-Fluoro-4-(morpholin-2-yl)phenyl)-6-methoxypyrazine-2-carboxamide hydrochloride

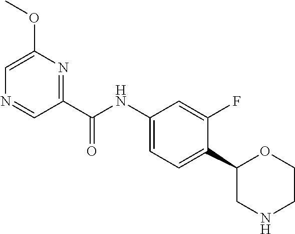 [SMILES: COc1cncc(C(=O)Nc2ccc([C@@H]3CNCCO3)c(F)c2)n1]

The title compound was obtained in analogy to example 1 using 6-methoxy-2-pyrazinecarboxylic acid (CAS 24005-61-6) in place of 5-(trifluoromethyl)pyrazine-2-carboxylic acid in step (i). Light yellow solid. MS (ISP): 333.4 ([M+H]⁺).

### Example 8

### (R)—N-(3-Fluoro-4-(morpholin-2-yl)phenyl)-5-methoxypyrazine-2-carboxamide hydrochloride

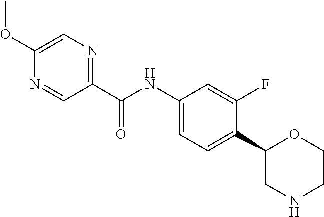 [SMILES: COc1cnc(C(=O)Nc2ccc([C@@H]3CNCCO3)c(F)c2)cn1]

The title compound was obtained in analogy to example 1 using 5-methoxy-2-pyrazinecarboxylic acid (CAS 40155-42-8) in place of 5-(trifluoromethyl)pyrazine-2-carboxylic acid in step (i). White solid. MS (ISP): 333.4 ([M+H]⁺).

### Example 9

### (S)—N-(3-Fluoro-4-(morpholin-2-yl)phenyl)-6-methoxypyrazine-2-carboxamide hydrochloride

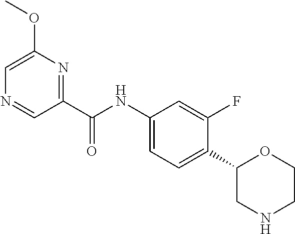 [SMILES: COc1cncc(C(=O)Nc2ccc([C@H]3CNCCO3)c(F)c2)n1]

The title compound was obtained in analogy to example 1 using (−)-(S)-2-(4-amino-2-fluoro-phenyl)-morpholine-4-carboxylic acid tert-butyl ester in place of (+)-(R)-2-(4-amino-2-fluoro-phenyl)-morpholine-4-carboxylic acid tert-butyl ester and 6-methoxy-2-pyrazinecarboxylic acid (CAS 24005-61-6) in place of 5-(trifluoromethyl)pyrazine-2-carboxylic acid in step (i). Light yellow solid. MS (ISP): 333.4 ([M+H]⁺).

### Example 10

### (S)—N-(3-Fluoro-4-(morpholin-2-yl)phenyl)-5-methoxypyrazine-2-carboxamide hydrochloride

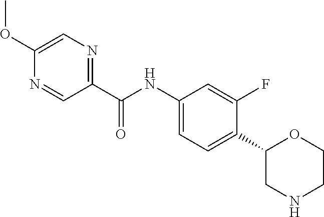 [SMILES: COc1cnc(C(=O)Nc2ccc([C@H]3CNCCO3)c(F)c2)cn1]

The title compound was obtained in analogy to example 1 using (−)-(S)-2-(4-amino-2-fluoro-phenyl)-morpholine-4-carboxylic acid tert-butyl ester in place of (+)-(R)-2-(4-amino-2-fluoro-phenyl)-morpholine-4-carboxylic acid tert-butyl ester and 5-methoxy-2-pyrazinecarboxylic acid (CAS 40155-42-8) in place of 5-(trifluoromethyl)pyrazine-2-carboxylic acid in step (i). White solid. MS (ISP): 333.4 ([M+H]⁺).

### Example 11

### (S)-5-(Cyclobutylmethoxy)-N-(3-fluoro-4-(morpholin-2-yl)phenyl)pyrazine-2-carboxamide hydrochloride

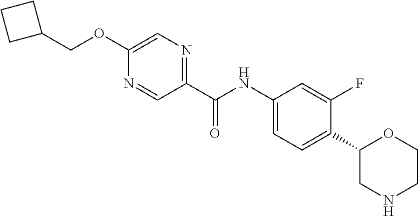 [SMILES: O=C(Nc1ccc([C@H]2CNCCO2)c(F)c1)c1cnc(OCC2CCC2)cn1]

### a) Ethyl 5-(cyclobutylmethoxy)-pyrazine-2-carboxylate

To a stirred solution of 2-carbethoxy-5-hydroxypyrazine (500 mg, CAS 54013-03-5) in THF (8 ml) were added cyclobutylmethanol (310 mg) and triphenylphosphine (936 mg). The resulting brown suspension was cooled to 0-5° C. and diisopropyl azodicarboxylate (747 μl) was added dropwise. The reaction mixture was stirred at RT for 3 hours. TLC analysis showed the reaction was complete. The reaction mixture was poured into sat. aq. NaHCO₃ and extracted twice with EtOAc. The combined organic layers were washed with sat. brine, then dried over Na₂SO₄ and concentrated in vacuo. The residue was purified by flash column chromatography (silica gel; eluent: 0% to 50% EtOAc in heptane) to afford ethyl 5-(cyclobutylmethoxy)-pyrazine-2-carboxylate (436 mg, 62%) as a white solid. MS (ISP): 237.5 ([M+H]⁺).

### b) 5-(Cyclobutylmethoxy)-pyrazine-2-carboxylic acid

To a stirred solution of ethyl 5-(cyclobutylmethoxy)-pyrazine-2-carboxylate (430 mg) in methanol (7 ml) was added 1 M aq. sodium hydroxide solution (7.28 ml) and the reaction mixture was stirred at RT for 1 hour. TLC analysis showed the reaction was complete. The reaction mixture was concentrated in vacuo then 25% aq. hydrochloric acid (3.93 ml) was added dropwise and the resulting mixture was then poured onto water and extracted twice with diethyl ether. The combined organic phases were dried over Na₂SO₄ and concentrated in vacuo to afford 5-(cyclobutylmethoxy)-pyrazine-2-carboxylic acid (343 mg, 91%) as a white solid which was used in the next step without further purification. MS (ISP): 207.5 ([M−H]⁻).

### c) (S)-5-(Cyclobutylmethoxy)-N-(3-fluoro-4-(morpholin-2-yl)phenyl)pyrazine-2-carboxamide hydrochloride

The title compound was obtained in analogy to example 1 using (−)-(S)-2-(4-amino-2-fluoro-phenyl)-morpholine-4-carboxylic acid tert-butyl ester in place of (+)-(R)-2-(4-amino-2-fluoro-phenyl)-morpholine-4-carboxylic acid tert-butyl ester and 5-(cyclobutylmethoxy)pyrazine-2-carboxylic acid in place of 5-(trifluoromethyl)pyrazine-2-carboxylic acid in step (i). White solid. MS (ISP): 387.2 ([M+H]⁺).

### Example 12

### (S)-5-(Cyclopropylmethoxy)-N-(3-fluoro-4-(morpholin-2-yl)phenyl)pyrazine-2-carboxamide hydrochloride

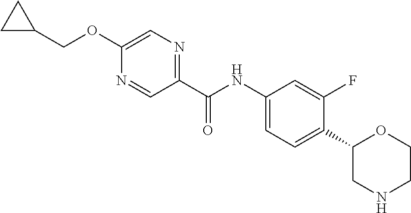 [SMILES: O=C(Nc1ccc([C@H]2CNCCO2)c(F)c1)c1cnc(OCC2CC2)cn1]

The title compound was obtained in analogy to example 1 using (−)-(S)-2-(4-amino-2-fluoro-phenyl)-morpholine-4-carboxylic acid tert-butyl ester in place of (+)-(R)-2-(4-amino-2-fluoro-phenyl)-morpholine-4-carboxylic acid tert-butyl ester and 5-(cyclopropylmethoxy)pyrazine-2-carboxylic acid (CAS 1286777-19-2) in place of 5-(trifluoromethyl)pyrazine-2-carboxylic acid in step (i). White solid. MS (ISP): 373.2 ([M+H]⁺).

### Example 13

### (S)-5-Ethoxy-N-(3-fluoro-4-(morpholin-2-yl)phenyl)pyrazine-2-carboxamide hydrochloride

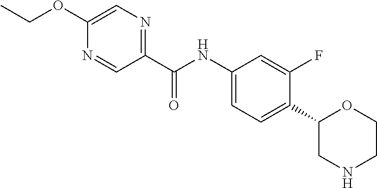 [SMILES: CCOc1cnc(C(=O)Nc2ccc([C@H]3CNCCO3)c(F)c2)cn1]

The title compound was obtained in analogy to example 1 using (−)-(S)-2-(4-amino-2-fluoro-phenyl)-morpholine-4-carboxylic acid tert-butyl ester in place of (+)-(R)-2-(4-amino-2-fluoro-phenyl)-morpholine-4-carboxylic acid tert-butyl ester and 5-ethoxy-2-pyrazinecarboxylic acid (CAS 1220330-11-9) in place of 5-(trifluoromethyl)pyrazine-2-carboxylic acid in step (i). White solid. MS (ISP): 347.2 ([M+H]⁺).

### Example 14

### (S)—N-(3-Fluoro-4-(morpholin-2-yl)phenyl)quinoxaline-2-carboxamide hydrochloride

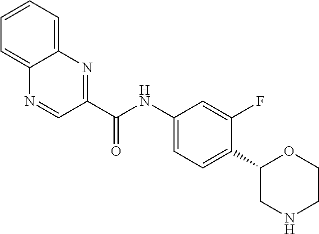 [SMILES: O=C(Nc1ccc([C@H]2CNCCO2)c(F)c1)c1cnc2ccccc2n1]

The title compound was obtained in analogy to example 1 using (−)-(S)-2-(4-amino-2-fluoro-phenyl)-morpholine-4-carboxylic acid tert-butyl ester in place of (+)-(R)-2-(4-amino-2-fluoro-phenyl)-morpholine-4-carboxylic acid tert-butyl ester and 2-quinoxalinecarboxylic acid (CAS 879-65-2) in place of 5-(trifluoromethyl)pyrazine-2-carboxylic acid in step (i). Light yellow solid. MS (ISP): 353.1 ([M+H]⁺).

### Example 15

### 7-Methyl-quinoxaline-2-carboxylic acid ((S)-3-fluoro-4-morpholin-2-yl-phenyl)-amide hydrochloride

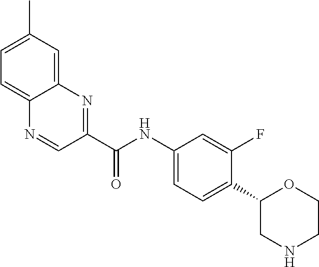 [SMILES: Cc1ccc2ncc(C(=O)Nc3ccc([C@H]4CNCCO4)c(F)c3)nc2c1]

The title compound was obtained in analogy to example 1 using (−)-(S)-2-(4-amino-2-fluoro-phenyl)-morpholine-4-carboxylic acid tert-butyl ester in place of (+)-(R)-2-(4-amino-2-fluoro-phenyl)-morpholine-4-carboxylic acid tert-butyl ester and 7-methyl-2-quinoxalinecarboxylic acid (CAS 14334-19-1) in place of 5-(trifluoromethyl)pyrazine-2-carboxylic acid in step (i). Off-white solid. MS (ISP): 367.6 ([M+H]⁺).

### Example 16

### 5-(3,3-Difluoro-azetidin-1-yl)-pyrazine-2-carboxylic acid ((S)-3-fluoro-4-morpholin-2-yl-phenyl)-amide hydrochloride

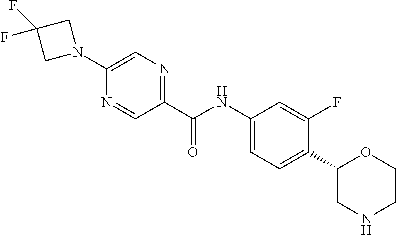 [SMILES: O=C(Nc1ccc([C@H]2CNCCO2)c(F)c1)c1cnc(N2CC(F)(F)C2)cn1]

### a) Methyl 5-(3,3-difluoroazetidin-1-yl)pyrazine-2-carboxylate

To a stirred solution of methyl 5-chloropyrazine-2-carboxylate (2.0 g, CAS 33332-25-1) in dioxane (45 ml) were added 3,3-difluoroazetidine hydrochloride (1.9 g, CAS 288315-03-7) and triethylamine (4.19 ml). The reaction mixture was stirred at 45° C. overnight. TLC analysis showed the reaction was complete. The reaction mixture was poured into aq. brine and extracted twice with ethyl acetate. The combined organic phases were dried over Na₂SO₄ and concentrated in vacuo. The residue was purified by flash column chromatography (silica gel; eluent: 50% ethyl acetate in heptane) to afford methyl 5-(3,3-difluoroazetidin-1-yl)pyrazine-2-carboxylate (1.21 g, 46%) as a white solid. MS (ISP): 230.2 ([M+H]⁺).

### b) 5-(3,3-Difluoroazetidin-1-yl)pyrazine-2-carboxylic acid

To a stirred solution of methyl 5-(3,3-difluoroazetidin-1-yl)pyrazine-2-carboxylate (600 mg) in tetrahydrofuran (10 ml) and water (5 ml) was added lithium hydroxide monohydrate (132 mg) and the reaction mixture was stirred at RT for 4 hours. TLC analysis showed the reaction was complete. 1 M aq. Hydrochloric acid (3.93 ml) was added dropwise and the reaction mixture was then poured onto water (15 ml) and extracted three times with ethyl acetate. The combined organic phases were dried over Na₂SO₄ and concentrated in vacuo to afford 5-(3,3-difluoroazetidin-1-yl)pyrazine-2-carboxylic acid (565 mg, quant.) as a white solid which was used in the next step without further purification. MS (ISP): 214 ([M−H]⁻).

### c) 5-(3,3-Difluoro-azetidin-1-yl)-pyrazine-2-carboxylic acid ((S)-3-fluoro-4-morpholin-2-yl-phenyl)-amide hydrochloride

The title compound was obtained in analogy to example 1 using (−)-(S)-2-(4-amino-2-fluoro-phenyl)-morpholine-4-carboxylic acid tert-butyl ester in place of (+)-(R)-2-(4-amino-2-fluoro-phenyl)-morpholine-4-carboxylic acid tert-butyl ester and 5-(3,3-difluoroazetidin-1-yl)pyrazine-2-carboxylic acid in place of 5-(trifluoromethyl)pyrazine-2-carboxylic acid in step (i). Off-white solid. MS (ISP): 394.5 ([M+H]⁺).

### Example 17

### 5-Cyclopropyl-pyrazine-2-carboxylic acid ((R)-3-fluoro-4-morpholin-2-yl-phenyl)-amide hydrochloride

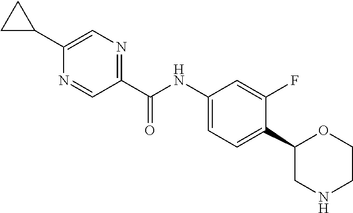 [SMILES: O=C(Nc1ccc([C@@H]2CNCCO2)c(F)c1)c1cnc(C2CC2)cn1]

The title compound was obtained in analogy to example 1 using 5-cyclopropyl-pyrazine-2-carboxylic acid (CAS 1211537-40-4) in place of 5-(trifluoromethyl)pyrazine-2-carboxylic acid in step (i). White solid. MS (ISP): 343.6 ([M+H]⁺).

### Example 18

### 5-Cyclopropyl-pyrazine-2-carboxylic acid ((S)-3-fluoro-4-morpholin-2-yl-phenyl)-amide hydrochloride

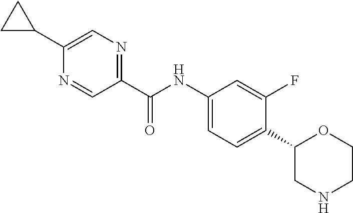 [SMILES: O=C(Nc1ccc([C@H]2CNCCO2)c(F)c1)c1cnc(C2CC2)cn1]

The title compound was obtained in analogy to example 1 using (−)-(S)-2-(4-amino-2-fluoro-phenyl)-morpholine-4-carboxylic acid tert-butyl ester in place of (+)-(R)-2-(4-amino-2-fluoro-phenyl)-morpholine-4-carboxylic acid tert-butyl ester and 5-cyclopropyl-pyrazine-2-carboxylic acid (CAS 1211537-40-4) in place of 5-(trifluoromethyl)pyrazine-2-carboxylic acid in step (i). White solid. MS (ISP): 343.6 ([M+H]⁺).

### Example 19

### (S)-5-Cyclopropyl-N-(2-fluoro-4-(morpholin-2-yl)phenyl)pyrazine-2-carboxamide

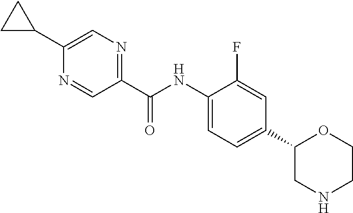 [SMILES: O=C(Nc1ccc([C@H]2CNCCO2)cc1F)c1cnc(C2CC2)cn1]

### a) 2-Chloro-1-(4-bromo-3-fluoro-phenyl)-ethanone

To a stirred solution of 4-bromo-3-fluorobenzoyl chloride (5.6 g, CAS 695188-21-7) in acetonitrile (30 ml) and THF (30 ml) at 0-5° C. was added dropwise (trimethylsilyl)diazomethane (13.7 ml, 2 M solution in diethyl ether). The reaction mixture was stirred at room temperature for 30 min. TLC analysis showed the reaction was complete. Hydrochloric acid (3.81 ml, 37% aq.) was then added dropwise at 0-5° C. over 10 minutes and the reaction mixture was then stirred at room temperature for a further 20 minutes. The reaction mixture was poured into EtOAc and extracted sequentially with aq. Na₂CO₃ solution, water and saturated brine. The organic layer was then dried over Na₂SO₄ and concentrated in vacuo to afford 2-chloro-1-(4-bromo-3-fluoro-phenyl)-ethanone (5.67 g) as a yellow solid which was used in the next step without further purification. MS (EI): 203 ([{⁸¹Br}M-CH₂Cl]⁺), 201 ([{⁷⁹Br}M-CH₂Cl]⁺), 175 ([{⁸¹Br}M-CH₂Cl—CO]⁺), 173 ([{⁷⁹Br}M-CH₂Cl—CO]⁺).

### b) (RS)-2-(4-Bromo-3-fluoro-phenyl)-oxirane

To a stirred solution of 2-chloro-1-(4-bromo-3-fluoro-phenyl)-ethanone (6.16 g) in ethanol (100 ml) at 5° C. was added portionwise over 5 min NaBH₄ (788 mg). The reaction mixture was then stirred at room temperature for 1 hour to afford a light yellow solution. TLC analysis showed the reaction was complete. Sodium methoxide (562 mg) was then added and the reaction mixture was stirred at room temperature overnight. TLC analysis showed a small amount of starting material remaining and so the reaction mixture was stirred at 40° C. for 1 h. The reaction mixture was then poured into water and extracted twice with EtOAc. The combined organic layers were washed with saturated brine, then dried over Na₂SO₄ and concentrated in vacuo to afford (RS)-2-(4-bromo-3-fluoro-phenyl)-oxirane (4.69 g) as a yellow oil which was used in the next step without further purification.

### c) (RS)-1-(4-Bromo-3-fluoro-phenyl)-2-(2-hydroxy-ethylamino)-ethanol

To a stirred solution of (RS)-2-(4-bromo-3-fluoro-phenyl)-oxirane (4.69 g) in THF (11 ml) was added 2-aminoethanol (13.2 ml) and the mixture was stirred at room temperature overnight. The reaction mixture was then poured into brine and extracted twice with EtOAc. The combined organic layers was dried over Na₂SO₄ and concentrated in vacuo to afford (RS)-1-(4-bromo-3-fluoro-phenyl)-2-(2-hydroxy-ethylamino)-ethanol (5.37 g) as a yellow viscous oil which was used in the next step without further purification. MS (ISP): 280.2 ([{⁸¹Br}M+H]⁺), 278.1 ([{⁷⁹Br}M+H]⁺).

### d) (RS)-[2-(4-Bromo-3-fluoro-phenyl)-2-hydroxy-ethyl]-(2-hydroxy-ethyl)-carbamic acid tert-butyl ester

To a stirred solution of (RS)-1-(4-bromo-3-fluoro-phenyl)-2-(2-hydroxy-ethylamino)-ethanol (5.37 g) in dichloromethane (60 ml) was added Boc₂O (4.00 g) and the mixture was stirred at room temperature overnight. The reaction mixture was then poured into water and extracted with dichloromethane. The organic layer was washed sequentially with 1 M aq. HCl, sat. aq. NaHCO₃ solution and saturated brine, then dried over Na2SO4 and concentrated in vacuo. The residue was purified by flash column chromatography (silica gel; gradient: 0% to 10% MeOH in dichloromethane) to afford (RS)-[2-(4-bromo-3-fluoro-phenyl)-2-hydroxy-ethyl]-(2-hydroxy-ethyl)-carbamic acid tert-butyl ester (3.89 g, 45% over 4 steps) as a light yellow viscous oil. MS (ISP): 380.1 ([{⁸¹Br}M+H]⁺), 378.2 ([{⁷⁹Br}M+H]⁺).

### e) (RS)-2-(4-Bromo-3-fluoro-phenyl)-morpholine-4-carboxylic acid tert-butyl ester

To a stirred solution of (RS)-[2-(4-bromo-3-fluoro-phenyl)-2-hydroxy-ethyl]-(2-hydroxy-ethyl)-carbamic acid tert-butyl ester (3.88 g) and triethylamine (1.71 ml) in THF (40 ml) at 0-5° C. was added dropwise methanesulfonyl chloride (873 μl). The reaction mixture was then stirred at room temperature for 30 min to afford a white suspension. The reaction mixture was then filtered to remove triethylamine hydrochloride, washing the filter with THF (6 ml). The filtrate was cooled to 0-5° C. and potassium 2-methyl-2-butoxide (9.05 ml, 1.7 M solution in toluene) was added. The reaction mixture was stirred at room temperature for 1 hour and then poured into water and extracted twice with EtOAc. The combined organic phases were dried over Na₂SO₄ and concentrated in vacuo. The residue was purified by flash column chromatography (silica gel; gradient: 0% to 30% EtOAc in hexanes) to afford (RS)-2-(4-bromo-3-fluoro-phenyl)-morpholine-4-carboxylic acid tert-butyl ester (1.73 g, 47%) as an orange viscous oil. MS (ISP): 306.1 ([{⁸¹Br}M+H—C₄H₈]⁺), 304.1 ([{⁷⁹Br}M+H—C₄H₈]⁺), 262.0 ([{⁸¹Br}M+H—C₄H₈—CO₂]⁺), 260.1 ([{⁷⁹Br}M+H—C₄H₈—CO₂]⁺).

### f) (RS)-2-[4-(Benzhydrylidene-amino)-3-fluoro-phenyl]-morpholine-4-carboxylic acid tert-butyl ester

To a stirred solution of (RS)-2-(4-bromo-3-fluoro-phenyl)-morpholine-4-carboxylic acid tert-butyl ester (1.57 g) and benzophenone imine (1.15 ml) in toluene (40 ml) was added sodium tert-butoxide (691 mg). The reaction mixture was purged with argon for 10 min. (R)-(+)-2,2′-bis(diphenylphosphino)-1,1′-binaphthyl (280 mg) and tris(dibenzylideneacetone)dipalladium(0) (120 mg) were added and the reaction mixture was heated to 100° C. and stirred for 1 h. The reaction mixture was poured into water and extracted twice with EtOAc. The organic layers were dried over Na₂SO₄ and concentrated in vacuo. The residue was purified by flash column chromatography (silica gel; gradient: 0% to 30% EtOAc in hexanes) to afford (RS)-2-[4-(benzhydrylidene-amino)-3-fluoro-phenyl]-morpholine-4-carboxylic acid tert-butyl ester (2.215 g, quant.) as a yellow viscous oil. MS (ISP): 461.3 ([M+H]⁺), 405.4 ([M+H—C₄H₈]⁺), 361.3 ([M+H—C₄H₈—CO₂]⁺).

### g) (RS)-2-(4-Amino-3-fluoro-phenyl)-morpholine-4-carboxylic acid tert-butyl ester

To a stirred solution of (RS)-2-[4-(benzhydrylidene-amino)-3-fluoro-phenyl]-morpholine-4-carboxylic acid tert-butyl ester (2.21 g) in methanol (40 ml) was added ammonium formate (4.54 g). The reaction mixture was degassed by bubbling argon into the mixture for several minutes. 10% Palladium on activated charcoal (255 mg) was then added and the reaction mixture was stirred at 60° C. for 1 hour. The reaction mixture was then filtered through celite and the filtrate was poured into 1 M aq. NaOH and extracted twice with EtOAc. The combined organic layers were dried over Na₂SO₄ and concentrated in vacuo. The residue was purified by flash column chromatography (silica gel; gradient: 0% to 30% EtOAc in hexanes) to afford (RS)-2-(4-amino-3-fluoro-phenyl)-morpholine-4-carboxylic acid tert-butyl ester (1.42 g, 74%) as a white solid. MS (ISP): 319.2 ([M+Na]⁺), 297.3 ([M+H]⁺), 241.2 ([M+H—C₄H₈]⁺), 197.2 ([M+H—C₄H₈—CO₂]⁺).

### h) (+)-(R)-2-(4-Amino-3-fluoro-phenyl)-morpholine-4-carboxylic acid tert-butyl ester & (−)-(S)-2-(4-Amino-3-fluoro-phenyl)-morpholine-4-carboxylic acid tert-butyl ester

The enantiomers of (RS)-2-(4-amino-3-fluoro-phenyl)-morpholine-4-carboxylic acid tert-butyl ester were separated using chiral HPLC (column: Chiralpak AD, 5×50 cm; eluent: 10% isopropanol/heptane; pressure: 18 bar; flow rate: 35 ml/min) affording:

(+)-(R)-2-(4-Amino-3-fluoro-phenyl)-morpholine-4-carboxylic acid tert-butyl ester (146 mg, light yellow solid), Retention time=62 min
(−)-(S)-2-(4-Amino-3-fluoro-phenyl)-morpholine-4-carboxylic acid tert-butyl ester (153 mg, off-white solid), Retention time=74 min

### i) (S)-2-(4-(5-Cyclopropylpyrazine-2-carboxamido)-3-fluorophenyl)morpholine-4-carboxylic acid tert-butyl ester

To a stirred suspension of (−)-(S)-2-(4-amino-3-fluoro-phenyl)-morpholine-4-carboxylic acid tert-butyl ester (140 mg) in THF (4 ml) and DMF (1 ml) were added sequentially N-methylmorpholine (208 μl), TBTU (303 mg) and 5-cyclopropyl-pyrazine-2-carboxylic acid (81 mg, CAS 1211537-40-4) and the mixture was heated at 50° C. overnight. TLC showed the reaction was complete. The mixture was then concentrated in vacuo and the residue was purified by column chromatography (SiO₂; gradient: 0% to 70% EtOAc in heptane) to give (S)-2-(4-(5-cyclopropylpyrazine-2-carboxamido)-3-fluorophenyl)morpholine-4-carboxylic acid tert-butyl ester (163 mg, 78%) as a white solid. MS (ISP): 460.3 ([M+NH₄]⁺).

### j) (S)-5-Cyclopropyl-N-(2-fluoro-4-(morpholin-2-yl)phenyl)pyrazine-2-carboxamide

To a stirred solution of trifluoroacetic acid (557 μl) in water (6 ml) was added a solution of (S)-2-(4-(5-cyclopropylpyrazine-2-carboxamido)-3-fluorophenyl)morpholine-4-carboxylic acid tert-butyl ester (160 mg) in acetonitrile (2 ml). The reaction mixture was then capped and the mixture was shaken at 80° C. overnight. The reaction mixture was then cooled to room temperature and poured into 2 M aq. NaOH and the resulting mixture was extracted twice with EtOAc. The organic layers were dried over Na₂SO₄ and concentrated in vacuo. The crude material was purified by flash column chromatography (Isolute® Flash-NH₂ from Separtis; gradient: MeOH/EtOAc/heptane) to afford (S)-5-cyclopropyl-N-(2-fluoro-4-(morpholin-2-yl)phenyl)pyrazine-2-carboxamide (90 mg, 73%) as a white solid. MS (ISP): 343.2 ([M+H]⁺).

### Example 20

### (R)-5-Cyclopropyl-N-(2-fluoro-4-(morpholin-2-yl)phenyl)pyrazine-2-carboxamide

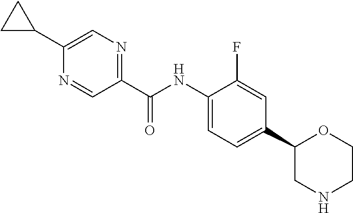 [SMILES: O=C(Nc1ccc([C@@H]2CNCCO2)cc1F)c1cnc(C2CC2)cn1]

The title compound was obtained in analogy to example 19 using (+)-(R)-2-(4-amino-3-fluoro-phenyl)-morpholine-4-carboxylic acid tert-butyl ester in place of (−)-(S)-2-(4-amino-3-fluoro-phenyl)-morpholine-4-carboxylic acid tert-butyl ester in step (i). White solid. MS (ISP): 343.2 ([M+H]⁺).

### Example 21

### 6-Isopropyl-pyrazine-2-carboxylic acid ((S)-3-fluoro-4-morpholin-2-yl-phenyl)-amide

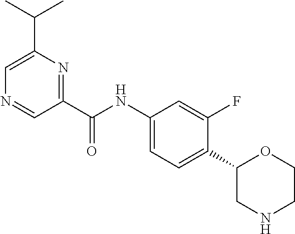 [SMILES: CC(C)c1cncc(C(=O)Nc2ccc([C@H]3CNCCO3)c(F)c2)n1]

The title compound was obtained in analogy to example 1 using (−)-(S)-2-(4-amino-2-fluoro-phenyl)-morpholine-4-carboxylic acid tert-butyl ester in place of (+)-(R)-2-(4-amino-2-fluoro-phenyl)-morpholine-4-carboxylic acid tert-butyl ester and 6-isopropyl-pyrazine-2-carboxylic acid (CAS 1302581-91-4) in place of 5-(trifluoromethyl)pyrazine-2-carboxylic acid in step (i). Colourless gum. MS (ISP): 345.6 ([M+H]⁺).

### Example 22

### 6-Isopropyl-pyrazine-2-carboxylic acid ((S)-2-fluoro-4-morpholin-2-yl-phenyl)-amide

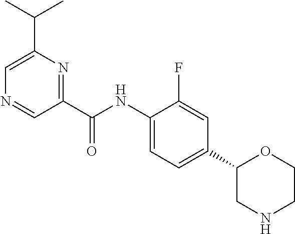 [SMILES: CC(C)c1cncc(C(=O)Nc2ccc([C@H]3CNCCO3)cc2F)n1]

The title compound was obtained in analogy to example 19 using 6-isopropyl-pyrazine-2-carboxylic acid (CAS 1302581-91-4) in place of 5-cyclopropyl-pyrazine-2-carboxylic acid in step (i). Colourless gum. MS (ISP): 345.6 ([M+H]⁺).

### Example 23

### (S)—N-(2-Fluoro-4-(morpholin-2-yl)phenyl)-5-(2,2,2-trifluoroethoxy)pyrazine-2-carboxamide

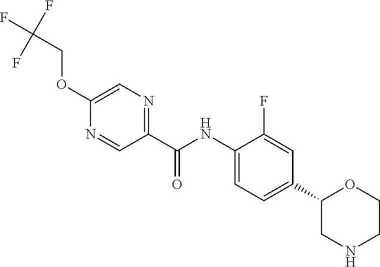 [SMILES: O=C(Nc1ccc([C@H]2CNCCO2)cc1F)c1cnc(OCC(F)(F)F)cn1]

The title compound was obtained in analogy to example 19 using 5-(2,2,2-trifluoroethoxy)pyrazine-2-carboxylic acid (CAS 1174323-36-4) in place of 5-cyclopropyl-pyrazine-2-carboxylic acid in step (i). White solid. MS (ISP): 401.1 ([M+H]⁺).

### Example 24

### (S)—N-(3-Fluoro-4-(morpholin-2-yl)phenyl)-5-(2,2,2-trifluoroethoxy)pyrazine-2-carboxamide

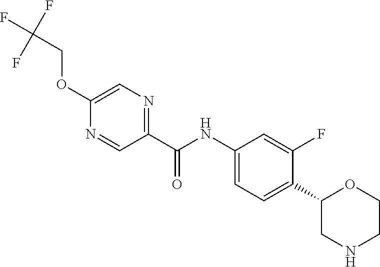 [SMILES: O=C(Nc1ccc([C@H]2CNCCO2)c(F)c1)c1cnc(OCC(F)(F)F)cn1]

The title compound was obtained in analogy to example 1 using (−)-(S)-2-(4-amino-2-fluoro-phenyl)-morpholine-4-carboxylic acid tert-butyl ester in place of (+)-(R)-2-(4-amino-2-fluoro-phenyl)-morpholine-4-carboxylic acid tert-butyl ester and 5-(2,2,2-trifluoroethoxy)pyrazine-2-carboxylic acid (CAS 1174323-36-4) in place of 5-(trifluoromethyl)pyrazine-2-carboxylic acid in step (i). White solid. MS (ISP): 401.1 ([M+H]⁺).

### Example 25

### (S)—N-(2-Fluoro-4-(morpholin-2-yl)phenyl)-6-(2,2,2-trifluoroethoxy)pyrazine-2-carboxamide

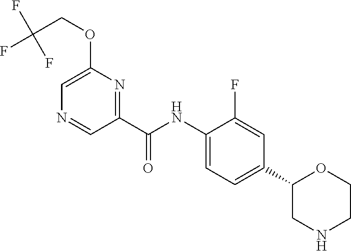 [SMILES: O=C(Nc1ccc([C@H]2CNCCO2)cc1F)c1cncc(OCC(F)(F)F)n1]

The title compound was obtained in analogy to example 19 using 6-(2,2,2-trifluoroethoxy)pyrazine-2-carboxylic acid (CAS 1346148-15-9) in place of 5-cyclopropyl-pyrazine-2-carboxylic acid in step (i). White solid. MS (ISP): 401.1 ([M+H]⁺).

### Example 26

### (S)—N-(3-Fluoro-4-(morpholin-2-yl)phenyl)-6-(2,2,2-trifluoroethoxy)pyrazine-2-carboxamide

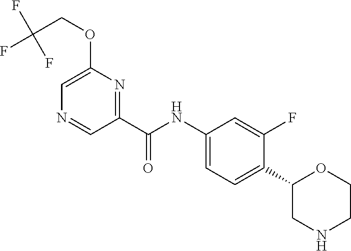 [SMILES: O=C(Nc1ccc([C@H]2CNCCO2)c(F)c1)c1cncc(OCC(F)(F)F)n1]

The title compound was obtained in analogy to example 1 using (−)-(S)-2-(4-amino-2-fluoro-phenyl)-morpholine-4-carboxylic acid tert-butyl ester in place of (+)-(R)-2-(4-amino-2-fluoro-phenyl)-morpholine-4-carboxylic acid tert-butyl ester and 6-(2,2,2-trifluoroethoxy)pyrazine-2-carboxylic acid (CAS 1346148-15-9) in place of 5-(trifluoromethyl)pyrazine-2-carboxylic acid in step (i). White solid. MS (ISP): 401.1 ([M+H]⁺).

### Example 27

### (R)—N-(2-Fluoro-4-(morpholin-2-yl)phenyl)-5-(2,2,2-trifluoroethoxy)pyrazine-2-carboxamide

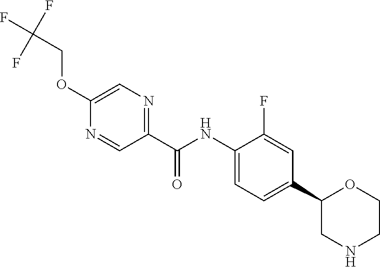 [SMILES: O=C(Nc1ccc([C@@H]2CNCCO2)cc1F)c1cnc(OCC(F)(F)F)cn1]

The title compound was obtained in analogy to example 19 using (+)-(R)-2-(4-amino-3-fluoro-phenyl)-morpholine-4-carboxylic acid tert-butyl ester in place of (−)-(S)-2-(4-amino-3-fluoro-phenyl)-morpholine-4-carboxylic acid tert-butyl ester and 5-(2,2,2-trifluoroethoxy)pyrazine-2-carboxylic acid (CAS 1174323-36-4) in place of 5-cyclopropyl-pyrazine-2-carboxylic acid in step (i). White solid. MS (ISP): 401.1 ([M+H]⁺).

### Example 28

### (R)—N-(3-Fluoro-4-(morpholin-2-yl)phenyl)-5-(2,2,2-trifluoroethoxy)pyrazine-2-carboxamide

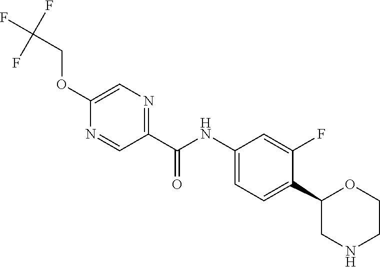 [SMILES: O=C(Nc1ccc([C@@H]2CNCCO2)c(F)c1)c1cnc(OCC(F)(F)F)cn1]

The title compound was obtained in analogy to example 1 using 5-(2,2,2-trifluoroethoxy)pyrazine-2-carboxylic acid (CAS 1174323-36-4) in place of 5-(trifluoromethyl)pyrazine-2-carboxylic acid in step (i). White solid. MS (ISP): 401.1 ([M+H]⁺).

### Example 29

### (R)—N-(2-Fluoro-4-(morpholin-2-yl)phenyl)-6-(2,2,2-trifluoroethoxy)pyrazine-2-carboxamide

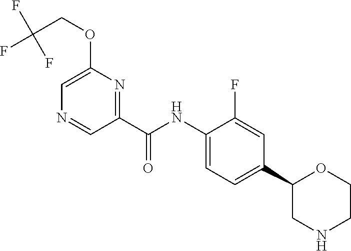 [SMILES: O=C(Nc1ccc([C@@H]2CNCCO2)cc1F)c1cncc(OCC(F)(F)F)n1]

The title compound was obtained in analogy to example 19 using (+)-(R)-2-(4-amino-3-fluoro-phenyl)-morpholine-4-carboxylic acid tert-butyl ester in place of (−)-(S)-2-(4-amino-3-fluoro-phenyl)-morpholine-4-carboxylic acid tert-butyl ester and 6-(2,2,2-trifluoroethoxy)pyrazine-2-carboxylic acid (CAS 1346148-15-9) in place of 5-cyclopropyl-pyrazine-2-carboxylic acid in step (i). White solid. MS (ISP): 401.1 ([M+H]⁺).

### Example 30

### (R)—N-(3-Fluoro-4-(morpholin-2-yl)phenyl)-6-(2,2,2-trifluoroethoxy)pyrazine-2-carboxamide

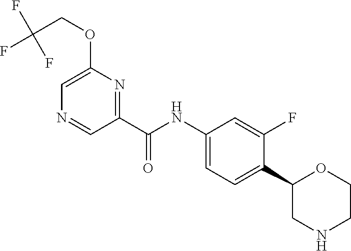 [SMILES: O=C(Nc1ccc([C@@H]2CNCCO2)c(F)c1)c1cncc(OCC(F)(F)F)n1]

The title compound was obtained in analogy to example 1 using 6-(2,2,2-trifluoroethoxy)pyrazine-2-carboxylic acid (CAS 1346148-15-9) in place of 5-(trifluoromethyl)pyrazine-2-carboxylic acid in step (i). White solid. MS (ISP): 401.1 ([M+H]⁺).

The compounds of formula I and their pharmaceutically usable addition salts possess valuable pharmacological properties. Specifically, it has been found that the compounds of the present invention have a good affinity to the trace amine associated receptors (TAARs), especially TAAR1.

The compounds were investigated in accordance with the test given hereinafter.

### Materials and Methods

### Construction of TAAR Expression Plasmids and Stably Transfected Cell Lines

For the construction of expression plasmids the coding sequences of human, rat and mouse TAAR 1 were amplified from genomic DNA essentially as described by Lindemann et al. [14]. The Expand High Fidelity PCR System (Roche Diagnostics) was used with 1.5 mM Mg²⁺ and purified PCR products were cloned into pCR2.1-TOPO cloning vector (Invitrogen) following the instructions of the manufacturer. PCR products were subcloned into the pIRESneo2 vector (BD Clontech, Palo Alto, Calif.), and expression vectors were sequence verified before introduction in cell lines.

HEK293 cells (ATCC # CRL-1573) were cultured essentially as described by Lindemann et al. (2005). For the generation of stably transfected cell lines HEK293 cells were transfected with the pIRESneo2 expression plasmids containing the TAAR coding sequences (described above) with Lipofectamine 2000 (Invitrogen) according to the instructions of the manufacturer, and 24 hrs post transfection the culture medium was supplemented with 1 mg/ml G418 (Sigma, Buchs, Switzerland). After a culture period of about 10 d clones were isolated, expanded and tested for responsiveness to trace amines (all compounds purchased from Sigma) with the cAMP Biotrak Enzyme immunoassay (EIA) System (Amersham) following the non-acetylation EIA procedure provided by the manufacturer. Monoclonal cell lines which displayed a stable EC₅₀ for a culture period of 15 passages were used for all subsequent studies.

### Radioligand Binding Assay on Rat TAAR1

### Membrane Preparation and Radioligand Binding.

HEK-293 cells stably expressing rat TAAR1 were maintained at 37° C. and 5% CO₂ in DMEM high glucose medium, containing fetal calf serum (10%, heat inactivated for 30 min at 56° C.), penicillin/streptomycin (1%), and 375 μg/ml geneticin (Gibco). Cells were released from culture flasks using trypsin/EDTA, harvested, washed twice with ice-cold PBS (without Ca²⁺ and Mg²⁺), pelleted at 1,000 rpm for 5 min at 4° C., frozen and stored at −80° C. Frozen pellets were suspended in 20 ml HEPES-NaOH (20 mM, pH 7.4) containing 10 mM EDTA and homogenized with a Polytron (PT 6000, Kinematica) at 14,000 rpm for 20 s. The homogenate was centrifuged at 48,000×g for 30 min at 4° C. Subsequently, the supernatant was removed and discarded, and the pellet resuspended in 20 ml HEPES-NaOH (20 mM, pH 7.4) containing 0.1 mM EDTA using the Polytron (20 s at 14,000 rpm). This procedure was repeated and the final pellet resuspended in HEPES-NaOH containing 0.1 mM EDTA and homogenized using the Polytron. Typically, aliquots of 2 ml membrane portions were stored at −80° C. With each new membrane batch the dissociation constant (K_{d}) was determined via a saturation curve. The TAAR1 radioligand ³[H]—(S)-4-[(ethyl-phenyl-amino)-methyl]-4,5-dihydro-oxazol-2-ylamine (described in WO 2008/098857) was used at a concentration equal to the calculated K_{d} value, that was usually around 2.3 nM, resulting in the binding of approximately 0.2% of the radioligand and a specific binding representing approximately 85% of the total binding. Nonspecific binding was defined as the amount of ³[H]—(S)-4-[(ethyl-phenyl-amino)-methyl]-4,5-dihydro-oxazol-2-ylamine bound in the presence of 10 μM unlabeled ligand. All compounds were tested at a broad range of concentrations (10 pM to 10 μM) in duplicates. The test compounds (20 μl/well) were transferred into a 96 deep well plate (TreffLab), and 180 μl of HEPES-NaOH (20 mM, pH 7.4) containing MgCl₂ (10 mM) and CaCl₂ (2 mM) (binding buffer), 300 μl of the radioligand ³[H]—(S)-4-[(ethyl-phenyl-amino)-methyl]-4,5-dihydro-oxazol-2-ylamine at a concentration of 3.3×K_{d} in nM and 500 μl of the membranes (resuspended at 50 μg protein per ml) added. The 96 deep well plates were incubated for 1 hr at 4° C. Incubations were terminated by rapid filtration through Unifilter-96 plates (Packard Instrument Company) and glass filters GF/C (Perkin Elmer) presoaked for 1 hr in polyethylenimine (0.3%) and washed 3 times with 1 ml of cold binding buffer. After addition of 45 μl of Microscint 40 (PerkinElmer) the Unifilter-96 plate was sealed and after 1 hr the ratio activity counted using a TopCount Microplate Scintillation Counter (Packard Instrument Company).

### Radioligand Binding Assay on Mouse TAAR1

### Membrane Preparation and Radioligand Binding.

HEK-293 cells stably expressing mouse TAAR1 were maintained at 37° C. and 5% CO₂ in DMEM high glucose medium, containing fetal calf serum (10%, heat inactivated for 30 min at 56° C.), penicillin/streptomycin (1%), and 375 μg/ml geneticin (Gibco). Cells were released from culture flasks using trypsin/EDTA, harvested, washed twice with ice-cold PBS (without Ca²⁺ and Mg²⁺), pelleted at 1,000 rpm for 5 min at 4° C., frozen and stored at −80° C. Frozen pellets were suspended in 20 ml HEPES-NaOH (20 mM, pH 7.4) containing 10 mM EDTA and homogenized with a Polytron (PT 6000, Kinematica) at 14,000 rpm for 20 s. The homogenate was centrifuged at 48,000×g for 30 min at 4° C. Subsequently, the supernatant was removed and discarded, and the pellet resuspended in 20 ml HEPES-NaOH (20 mM, pH 7.4) containing 0.1 mM EDTA using the Polytron (20 s at 14,000 rpm). This procedure was repeated and the final pellet resuspended in HEPES-NaOH containing 0.1 mM EDTA and homogenized using the Polytron. Typically, aliquots of 2 ml membrane portions were stored at −80° C. With each new membrane batch the dissociation constant (K_{d}) was determined via a saturation curve. The TAAR1 radioligand ³[H]—(S)-4-[(ethyl-phenyl-amino)-methyl]-4,5-dihydro-oxazol-2-ylamine (described in WO 2008/098857) was used at a concentration equal to the calculated K_{d} value, that was usually around 0.7 nM, resulting in the binding of approximately 0.5% of the radioligand and a specific binding representing approximately 70% of the total binding. Nonspecific binding was defined as the amount of ³[H]—(S)-4-[(ethyl-phenyl-amino)-methyl]-4,5-dihydro-oxazol-2-ylamine bound in the presence of 10 μM unlabeled ligand. All compounds were tested at a broad range of concentrations (10 pM to 10 μM) in duplicates. The test compounds (20 μl/well) were transferred into a 96 deep well plate (TreffLab), and 180 μl of HEPES-NaOH (20 mM, pH 7.4) containing MgCl₂ (10 mM) and CaCl₂ (2 mM) (binding buffer), 300 μl of the radioligand ³[H]—(S)-4-[(ethyl-phenyl-amino)-methyl]-4,5-dihydro-oxazol-2-ylamine at a concentration of 3.3×K_{d} in nM and 500 μl of the membranes (resuspended at 60 μg protein per ml) added. The 96 deep well plates were incubated for 1 hr at 4° C. Incubations were terminated by rapid filtration through Unifilter-96 plates (Packard Instrument Company) and glass filters GF/C (Perkin Elmer) presoaked for 1 hr in polyethylenimine (0.3%) and washed 3 times with 1 ml of cold binding buffer. After addition of 45 μl of Microscint 40 (PerkinElmer) the Unifilter-96 plate was sealed and after 1 hr the radioactivity counted using a TopCount Microplate Scintillation Counter (Packard Instrument Company).

The compounds show a Kᵢ value (μM) in mouse or rat on TAAR1 (in μM) as shown in the table below.

| | Ki (μM) |
|---|---|
| Example | mouse/rat |
| 1 | 0.0115/0.009 |
| 2 | 0.0184/0.0186 |
| 3 | 0.0204/1.0905 |
| 4 | 0.0833/0.0344 |
| 5 | 0.038/0.054 |
| 6 | 0.0328/0.0197 |
| 7 | 0.1469/0.068 |
| 8 | 0.0107/0.0476 |
| 9 | 0.0314/0.247 |
| 10 | 0.0136/0.5397 |
| 11 | 0.0046/0.0119 |
| 12 | 0.0045/0.04 |
| 13 | 0.0041/0.1102 |
| 14 | 0.0028/0.0222 |
| 15 | 0.0068/0.0131 |
| 16 | 0.0193/0.1906 |
| 17 | 0.0072/0.0076 |
| 18 | 0.0036/0.0625 |
| 19 | 0.0061/0.2348 |
| 20 | 0.0099/0.0708 |
| 21 | 0.0181/0.0849 |
| 22 | 0.0522/0.3913 |
| 23 | 0.0046/0.0881 |
| 24 | 0.0042/0.0202 |
| 25 | 0.044/0.0122 |
| 26 | 0.0167/0.0032 |
| 27 | 0.0059/0.0183 |
| 28 | 0.0053/0.0037 |
| 29 | 0.0447/0.0053 |
| 30 | 0.0292/0.0035 |

The compounds of formula I and the pharmaceutically acceptable salts of the compounds of formula I can be used as medicaments, e.g. in the form of pharmaceutical preparations. The pharmaceutical preparations can be administered orally, e.g. in the form of tablets, coated tablets, dragées, hard and soft gelatine capsules, solutions, emulsions or suspensions. The administration can, however, also be effected rectally, e.g. in the form of suppositories, or parenterally, e.g. in the form of injection solutions.

The compounds of formula I can be processed with pharmaceutically inert, inorganic or organic carriers for the production of pharmaceutical preparations. Lactose, corn starch or derivatives thereof, talc, stearic acids or its salts and the like can be used, for example, as such carriers for tablets, coated tablets, dragées and hard gelatine capsules. Suitable carriers for soft gelatine capsules are, for example, vegetable oils, waxes, fats, semi-solid and liquid polyols and the like. Depending on the nature of the active substance no carriers are however usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical preparations can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Medicaments containing a compound of formula I or a pharmaceutically acceptable salt thereof and a therapeutically inert carrier are also an object of the present invention, as is a process for their production, which comprises bringing one or more compounds of formula I and/or pharmaceutically acceptable acid addition salts and, if desired, one or more other therapeutically valuable substances into a galenical administration form together with one or more therapeutically inert carriers.

The most preferred indications in accordance with the present invention are those which include disorders of the central nervous system, for example the treatment or prevention of depression, psychosis, Parkinson's disease, anxiety, attention deficit hyperactivity disorder (ADHD) and diabetes.

The dosage can vary within wide limits and will, of course, have to be adjusted to the individual requirements in each particular case. In the case of oral administration the dosage for adults can vary from about 0.01 mg to about 1000 mg per day of a compound of general formula I or of the corresponding amount of a pharmaceutically acceptable salt thereof. The daily dosage may be administered as single dose or in divided doses and, in addition, the upper limit can also be exceeded when this is found to be indicated.

Tablet Formulation (Wet Granulation)

| | | mg/tablet | | | |
|---|---|---|---|---|---|
| Item | Ingredients | 5 mg | 25 mg | 100 mg | 500 mg |
| 1. | Compound of formula I | 5 | 25 | 100 | 500 |
| 2. | Lactose Anhydrous DTG | 125 | 105 | 30 | 150 |
| 3. | Sta-Rx 1500 | 6 | 6 | 6 | 30 |
| 4. | Microcrystalline Cellulose | 30 | 30 | 30 | 150 |
| 5. | Magnesium Stearate | 1 | 1 | 1 | 1 |
| | Total | 167 | 167 | 167 | 831 |

### Manufacturing Procedure

1. Mix items 1, 2, 3 and 4 and granulate with purified water.
2. Dry the granules at 50° C.
3. Pass the granules through suitable milling equipment.
4. Add item 5 and mix for three minutes; compress on a suitable press.

Capsule Formulation

| | | mg/capsule | | | |
|---|---|---|---|---|---|
| Item | Ingredients | 5 mg | 25 mg | 100 mg | 500 mg |
| 1. | Compound of formula I | 5 | 25 | 100 | 500 |
| 2. | Hydrous Lactose | 159 | 123 | 148 | — |
| 3. | Corn Starch | 25 | 35 | 40 | 70 |
| 4. | Talc | 10 | 15 | 10 | 25 |
| 5. | Magnesium Stearate | 1 | 2 | 2 | 5 |
| | Total | 200 | 200 | 300 | 600 |

### Manufacturing Procedure

1. Mix items 1, 2 and 3 in a suitable mixer for 30 minutes.
2. Add items 4 and 5 and mix for 3 minutes.
3. Fill into a suitable capsule.

1. A compound of formula 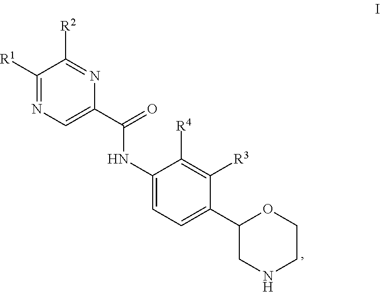
wherein
R¹/R² are hydrogen, lower alkyl, lower alkoxy, lower alkyl substituted by halogen, lower alkoxy substituted by halogen, cycloalkyl, OCH₂-cycloalkyl or heterocycloalkyl which is optionally substituted by halogen, with the proviso that one of R¹ and R² is hydrogen, or R¹ and R² form together with the carbon atom to which they are attach a phenyl ring, which may be optionally substituted by lower alkyl;
R³/R⁴ are hydrogen, halogen or cyano;
with the proviso that one of R³ and R⁴ is hydrogen;
or a pharmaceutically suitable acid addition salt thereof, all racemic mixtures, all their corresponding enantiomers and/or optical isomers.

2. A compound of formula I according to claim 1, wherein “halogen” is fluorine.

3. A compound of formula I according to claim 1, wherein R¹ is lower alkyl, lower alkoxy, lower alkyl substituted by halogen, lower alkoxy substituted by halogen, cycloalkyl, OCH₂-cycloalkyl or heterocycloalkyl which is optionally substituted by halogen, and R² is hydrogen.

4. A compound of formula I according to claim 3, which compounds are
(R)—N-(3-fluoro-4-(morpholin-2-yl)phenyl)-5-(trifluoromethyl)pyrazine-2-carboxamide
(R)—N-(3-cyano-4-(morpholin-2-yl)phenyl)-5-(trifluoromethyl)pyrazine-2-carboxamide
(S)—N-(3-cyano-4-(morpholin-2-yl)phenyl)-5-(trifluoromethyl)pyrazine-2-carboxamide
(R)—N-(3-fluoro-4-(morpholin-2-yl)phenyl)-5-methoxypyrazine-2-carboxamide
(S)—N-(3-fluoro-4-(morpholin-2-yl)phenyl)-5-methoxypyrazine-2-carboxamide
(S)-5-(cyclobutylmethoxy)-N-(3-fluoro-4-(morpholin-2-yl)phenyl)pyrazine-2-carboxamide
(S)-5-(cyclopropylmethoxy)-N-(3-fluoro-4-(morpholin-2-yl)phenyl)pyrazine-2-carboxamide
(S)-5-ethoxy-N-(3-fluoro-4-(morpholin-2-yl)phenyl)pyrazine-2-carboxamide
5-(3,3-difluoro-azetidin-1-yl)-pyrazine-2-carboxylic acid ((S)-3-fluoro-4-morpholin-2-yl-phenyl)-amide
(R)-5-cyclopropyl-N-(2-fluoro-4-(morpholin-2-yl)phenyl)pyrazine-2-carboxamide
5-cyclopropyl-pyrazine-2-carboxylic acid ((R)-3-fluoro-4-morpholin-2-yl-phenyl)-amide
5-cyclopropyl-pyrazine-2-carboxylic acid ((S)-3-fluoro-4-morpholin-2-yl-phenyl)-amide
(S)-5-cyclopropyl-N-(2-fluoro-4-(morpholin-2-yl)phenyl)pyrazine-2-carboxamide
(R)-5-cyclopropyl-N-(2-fluoro-4-(morpholin-2-yl)phenyl)pyrazine-2-carboxamide
(S)—N-(2-Fluoro-4-(morpholin-2-yl)phenyl)-5-(2,2,2-trifluoroethoxy)pyrazine-2-carboxamide
(S)—N-(3-Fluoro-4-(morpholin-2-yl)phenyl)-5-(2,2,2-trifluoroethoxy)pyrazine-2-carboxamide
(R)—N-(2-Fluoro-4-(morpholin-2-yl)phenyl)-5-(2,2,2-trifluoroethoxy)pyrazine-2-carboxamide or
(R)—N-(3-Fluoro-4-(morpholin-2-yl)phenyl)-5-(2,2,2-trifluoroethoxy)pyrazine-2-carboxamide.

5. A compound of formula I according to claim 1, wherein R² is lower alkyl, lower alkoxy, lower alkyl substituted by halogen, lower alkoxy substituted by halogen, cycloalkyl, OCH₂-cycloalkyl or heterocycloalkyl which is optionally substituted by halogen, and R¹ is hydrogen.

6. A compound of formula I according to claim 5, which compounds are
(R)—N-(3-cyano-4-(morpholin-2-yl)phenyl)-6-(trifluoromethyl)pyrazine-2-carboxamide
(S)—N-(3-fluoro-4-(morpholin-2-yl)phenyl)-6-(trifluoromethyl)pyrazine-2-carboxamide
(R)—N-(3-fluoro-4-(morpholin-2-yl)phenyl)-6-(trifluoromethyl)pyrazine-2-carboxamide
(R)—N-(3-fluoro-4-(morpholin-2-yl)phenyl)-6-methoxypyrazine-2-carboxamide
(S)—N-(3-fluoro-4-(morpholin-2-yl)phenyl)-6-methoxypyrazine-2-carboxamide
6-Isopropyl-pyrazine-2-carboxylic acid ((S)-3-fluoro-4-morpholin-2-yl-phenyl)-amide
6-Isopropyl-pyrazine-2-carboxylic acid ((S)-2-fluoro-4-morpholin-2-yl-phenyl)-amide
(S)—N-(2-Fluoro-4-(morpholin-2-yl)phenyl)-6-(2,2,2-trifluoroethoxy)pyrazine-2-carboxamide
(S)—N-(3-Fluoro-4-(morpholin-2-yl)phenyl)-6-(2,2,2-trifluoroethoxy)pyrazine-2-carboxamide
(R)—N-(2-Fluoro-4-(morpholin-2-yl)phenyl)-6-(2,2,2-trifluoroethoxy)pyrazine-2-carboxamide or
(R)—N-(3-Fluoro-4-(morpholin-2-yl)phenyl)-6-(2,2,2-trifluoroethoxy)pyrazine-2-carboxamide.

7. A compound of formula I according to claim 1, wherein R¹ and R² form together with the carbon atom to which they are attached a phenyl ring, which may be optionally substituted by lower alkyl.

8. A compound of formula I according to claim 7, which compounds are
(S)—N-(3-fluoro-4-(morpholin-2-yl)phenyl)quinoxaline-2-carboxamide or
7-methyl-quinoxaline-2-carboxylic acid ((S)-3-fluoro-4-morpholin-2-yl-phenyl)-amide.

9. A process for the manufacture of a compound of formula I as defined in claim 1, which process comprises
a) cleaving off the N-protecting group (PG) from compounds of formula 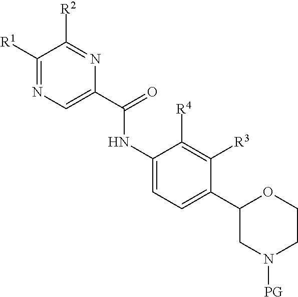
to a compound of formula 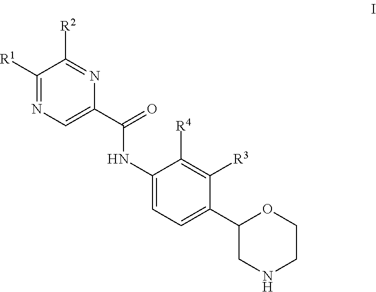
wherein PG is a N-protecting group selected from —C(O)O-tert-butyl and the other definitions are as described in claim 1, and,
if desired, converting the compounds obtained into pharmaceutically acceptable acid addition salts.

10. A compound manufactured by a process according to claim 9.

11. A pharmaceutical composition comprising a compound according to claim 1 and a pharmaceutical acceptable carrier and/or adjuvant.

12.-14. (canceled)

15. A method for the therapeutic and/or prophylactic treatment of a disease or disorder selected from the group consisting of depression, anxiety disorders, bipolar disorder, attention deficit hyperactivity disorder (ADHD), stress-related disorders, psychotic disorders, schizophrenia, neurological diseases, Parkinson's disease, neurodegenerative disorders, Alzheimer's disease, epilepsy, migraine, hypertension, substance abuse, metabolic disorders, eating disorders, diabetes, diabetic complications, obesity, dyslipidemia, disorders of energy consumption and assimilation, disorders and malfunction of body temperature homeostasis, disorders of sleep and circadian rhythm, and cardiovascular disorders, the method comprising the administration of a therapeutically effective amount of a compound according to claim 1.

16. (canceled)
